(19) United States
(12) Patent Application Publication
(10) Pub. No.: US 2004/0137510 A1
(43) Pub. Date: Jul. 15, 2004

(54) Method for detecting growth hormone variations in humans, the variations and their uses

(75) Inventors: David N. Cooper, Cardiff (GB); Annie M. Procter, Cardiff (GB); John Gregory, Cardiff (GB); David S. Millar, Cardiff (GB)

Correspondence Address: YOUNG & THOMPSON, 745 SOUTH 23RD STREET 2ND FLOOR, ARLINGTON, VA, 22202

(73) Assignee: UNIVERSITY OF WALES COLLEGE OF MEDICINE, CARDIFF (GB)

(21) Appl. No.: 10/788,318

(22) Filed: Mar. 1, 2004

Related U.S. Application Data
(62) Division of application No. 09/853,688, filed on May 14, 2001.

(30) Foreign Application Priority Data
May 12, 2000 (GB) 0011459.5
Jul. 14, 2000 (EP) 00306004.3

(51) Int. Cl. C12Q 1/68

(52) U.S. Cl. 435/6

(57) ABSTRACT

A detection method for detecting a variation in GH1 effective to act as an indicator of GH dysfunction in an individual, comprises the steps of comparing a test sample comprising a nucleotide sequence of the human GH1 gene from the individual with a standard sequence known to be that of the human GH1 gene. A difference between the test sample sequence and the standard sequence indicates the presence of a variation effective to act as an indicator of GH dysfunction (hereinafter “variant of GH1”). The test sample is obtained from a individual exhibiting the following criterion:

(i) growth failure, defined as a growth pattern [delineated by a series of height measurements; Brook CDG (Ed) Clinical Paediatric Endocrinology 3rd Ed, Chapter 9, pl41 (1995, Blackwell Science)] which, when plotted on a standard height chart [Tanner et al Arch Dis Child 45 755-762 (1970)], predicts an adult height for the individual which is outside the individual's estimated target adult height range, the estimate being based upon the heights of the individual's parents.

Also disclosed are mutations thereby detected, and their use in screening patients for growth hormone irregularities or for producing variant proteins suitable for treating such irregularities.

The present invention relates to a method for detecting naturally-occurring growth hormone mutations; to mutations thereby detected and their use in screening patients for growth hormone irregularities or for producing variant proteins suitable for treating such irregularities.

That human stature was influenced by inherited factors was understood more than a century ago. Although familial short stature, with its normally recessive mode of inheritance, was recognised as early as 1912, it was a further quarter century before such families came to be properly documented in the scientific literature. The recognition that recessively inherited short stature was commonly associated with isolated growth hormone (GH) deficiency only came in 1966.

Short stature associated with GH deficiency has been estimated to occur with an incidence of between 1/4000 and 1/10000 live births. Most of these cases are both sporadic and idiopathic, but between 5 and 30% have an affected first-degree relative consistent with a genetic aetiology for the condition. Confirmation of the genetic aetiology of GH deficiency came from the molecular genetic analysis of familial short stature and the early demonstration of mutational lesions in the pituitary-expressed growth hormone (GHI) genes of affected individuals. Familial short stature may also be caused by mutation in a number of other genes (eg POUlFl, PROPl and GHRHR) and it is important to distinguish these different forms of the condition.

Growth hormone (GH) is a multifunctional hormone that promotes post-natal growth of skeletal and soft tissues through a variety of effects. Controversy remains as to the relative contribution of direct and indirect actions of GH. On one hand, the direct effects of GH have been demonstrated in a variety of tissues and organs, and GH receptors have been documented in a number of cell types. On the other hand, a substantial amount of data indicates that a major portion of the effects of GH are mediated through the actions of GH-dependent insulin-like growth factor I (IGF-I). IGF-I is produced in many tissues, primarily the liver, and acts through its own receptor to enhance the proliferation and maturation of many tissues, including bone, cartilage, and skeletal muscle. In addition to promoting growth of tissues, GH has also been shown to exert a variety of other biological effects, including lactogenic, diabetogenic, lipolytic and protein anabolic effects, as well as sodium and water retention.

Adequate amounts of GH are needed throughout childhood to maintain normal growth. Newborns with GH deficiency are usually of normal length and weight. Some may have a micropenis or fasting hypoglycemia in conjunction with low linear postnatal growth, which becomes progressively retarded with age. In those with isolated growth hormone deficiency (IGHD), skeletal maturation is usually delayed in association with their height retardation. Truncal obesity, facial appearance younger than expected for their chronological age and delayed secondary dentition are often present. Skin changes similar to those seen in premature ageing may be seen in affected adults.

Familial IGHD comprises several different disorders with characteristic modes of inheritance. Those forms of IGHD known to be associated with defects at the GH1 gene locus are shown in Table 1 together with the different types of underlying lesion so far detected.

TABLE 1
Classification of inherited disorders involving the GH1 gene

| | Mode of | Types of gene lesion | GH | |
|---|---|---|---|---|
| Disorder | inheritance | responsible | protein | Deficiency state |
| IGHD IA | Autosomal | Gross deletions, | Absent | Severe short stature. Anti-GH |
| | recessive | micro-deletions, | | antibodies often produced |
| | | nonsense mutations | | upon GH treatment, resulting |
| | | | | in poor response thereto. |
| IGHD IB | Autosomal | Splice site mutations | Deficient | Short stature. Patients usually |
| | recessive | | | respond well to exogenous |
| | | | | GH. |
| IGHD II | Autosomal | Splice site and | Deficient | Short stature. Patients usually |
| | dominant | intronic mutations, | | respond well to exogenous |
| | | missense mutations | | GH. |

The characterisation of these lesions has helped to provide explanations for the differences in clinical severity, mode of inheritance and propensity to antibody formation in response to exogenously administered GH, between these forms of IGHD. Most cases are sporadic and are assumed to arise from cerebral insults or defects that include cerebral oedema, chromosomal anomalies, histiocytosis, infections, radiation, septo-optic dysplasia, trauma, or tumours affecting the hypothalamus or pituitary. Magnetic resonance imaging examinations detect hypothalamic or pituitary anomalies in about 12% of patients who have IGHD.

Although short stature, delayed ‘height velocity’ or growth velocity, and delayed skeletal maturation are all seen with GH deficiency, none of these is specific for this disorder; other systemic diseases may result in such symptoms. Throughout this specification, ‘height velocity’ and growth velocity are both to be construed as meaning the rate of change of the subject's or patient's height, such as is measured in centimetres per year.

Stimulation tests to demonstrate GH deficiency use L-Dopa, insulin-induced hypoglycaemia, arginine, insulin-arginine, clonidine, glucagon or propranolol.

Inadequate GH peak responses (usually <7-10 ng/mL) differ from test to test. Testing for concomitant deficiencies of LH, FSH, TSH and ACTH should be performed to determine the extent of pituitary dysfunction and to plan optimal treatment.

Recombinant-derived GH is available worldwide and is administered by subcutaneous injection. To obtain an optimal outcome, children with IGHD are usually started on replacement therapy as soon as their diagnosis is established. The initial dosage of recombinant GH is based on body weight or surface area, but the exact amount used and the frequency of administration may vary between different protocols. The dosage increases with increasing body weight to a maximum during puberty. Thereafter, GH treatment should be temporarily discontinued while the individual's GH secretory capacity is re-evaluated. Those with confirmed GH deficiency receive a lower dose of exogenous GH during adult life.

Conditions that are treated with GH include (i) those in which it has proven efficacy and (ii) a variety of others in which its use has been reported but not accepted as standard practice. Disorders in which GH treatment has proven efficacy include GH deficiency, either isolated or in association with combined pituitary hormone deficiency (CPHD) and Turner syndrome. The clinical responses of individuals with the first two disorders to GH replacement therapy varies depending on: (i) the severity of the GH deficiency and its adverse effects on growth, the age at which treatment is begun, weight at birth, current weight and dose of GH; and (ii) recognition and response to treatment of associated deficiencies such as thyroid hormone deficiency; and (iii) whether treatment is complicated by the development of anti-GH antibodies. The outcome of treatment for individuals with Turner syndrome varies with the severity of their short stature, their chromosomal complement, and the age at which treatment was begun.

Additional disorders in which the use of GH has been reported include treatment of certain skeletal dysplasias such as achondroplasia, Prader-Willi syndrome, growth suppression secondary to exogenous steroids or in association with chronic inflammatory diseases such as rheumatoid arthritis, in chronic renal failure, extreme idiopathic short stature, Russell-Silver syndrome, and intrauterine growth retardation.

The characterisation of familial IGHD at the molecular genetic level is important for several reasons. The identity of the locus involved will indicate not only the likely severity of growth retardation but, more importantly, the appropriateness or otherwise of the various therapeutic regimens now available. Further, detection of the underlying gene lesions serves to confirm the genetic aetiology of the condition. It may also have prognostic value in predicting (i) the severity of growth retardation and (ii) the likelihood of anti-GH antibody formation subsequent to GH treatment. In some instances, knowledge of the pathological lesion(s) can also help to explain an unusual mode of inheritance of the disorder and is therefore essential for the counselling of affected families. Finally, the characterisation of the mutational lesions responsible for cases of IGHD manifesting a dysfunctional (as opposed to a non-functional) GH molecule could yield new insights into GH structure and function.

At the cellular level, a single GH molecule binds two GH receptor molecules (GHR) causing them to dimerise. Dimerisation of the two GH-bound GHR molecules is believed to be necessary for signal transduction, which is associated with the tyrosine kinase JAK-2. It has been suggested that the diverse effects of GH may be mediated by a single type of GHR molecule that can possess different cytoplasmic domains or phosphorylation sites in different tissues. When activated by JAK-2, these differing cytoplasmic domains can lead to distinct phosphorylation pathways one for growth effects and others for various metabolic effects.

GH is a 22 kDa protein secreted by the somatotroph cells of the anterior pituitary. X-ray crystallographic studies have shown GH to comprise a core of two pairs of parallel alpha helices arranged in an up-up-down-down fashion. This structure is stabilised by two intra-molecular disulphide linkages (Cys53-Cys165 and Cysl82-Cys 189). Two growth hormone receptor (GHR) molecules bind to two structurally distinct sites on the GH molecule, a process which proceeds sequentially by GHR binding first at site 1 and then at site 2. The binding of GHR to GH potentiates dimerisation of the GHR molecules.

Scanning mutagenesis studies of the GH molecule have yielded a picture of the binding interactions between GH and its receptor whilst site-directed mutagenesis has been used to probe the fulction of specific residues. Thus, substitution of Gly120 (in the third alpha helix of human GH) by Arg results in the loss of GHR binding to site 2 thereby blocking GHR dimerisation. Similarly, residue Phe44 of the human GH protein is important for binding the prolactin receptor. Finally, residues Asp115, Gly119, Ala122 and Leu123 have been shown to be critical for the growth enhancing potential of the murine GH molecule.

Interaction of the dimerised GHR with the intracellular tyrosine protein kinase JAY2 leads to tyrosine phosphorylation of downstream signal transduction molecules, stimulation of mitogen-activated protein (MAP) kinases and induction of signal transducers and activators of transcription (STAT proteins). In this way, GH is able to influence the expression of multiple genes through a number of different signalling pathways.

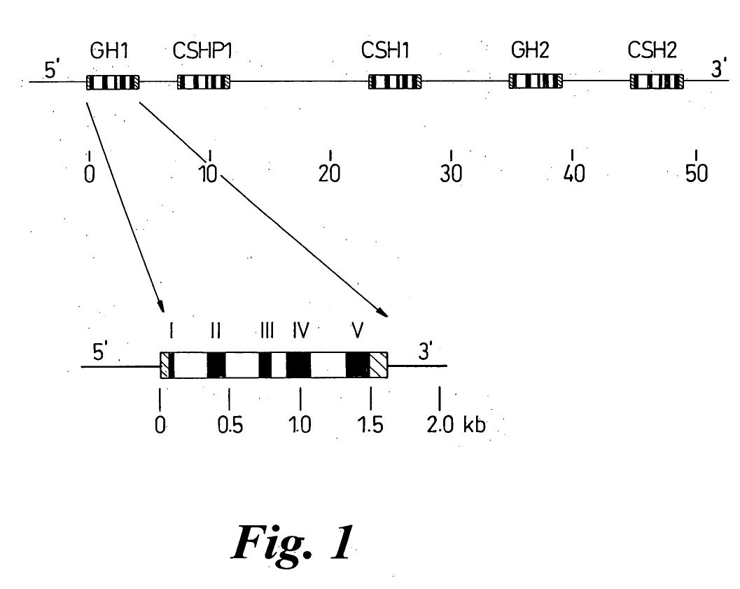

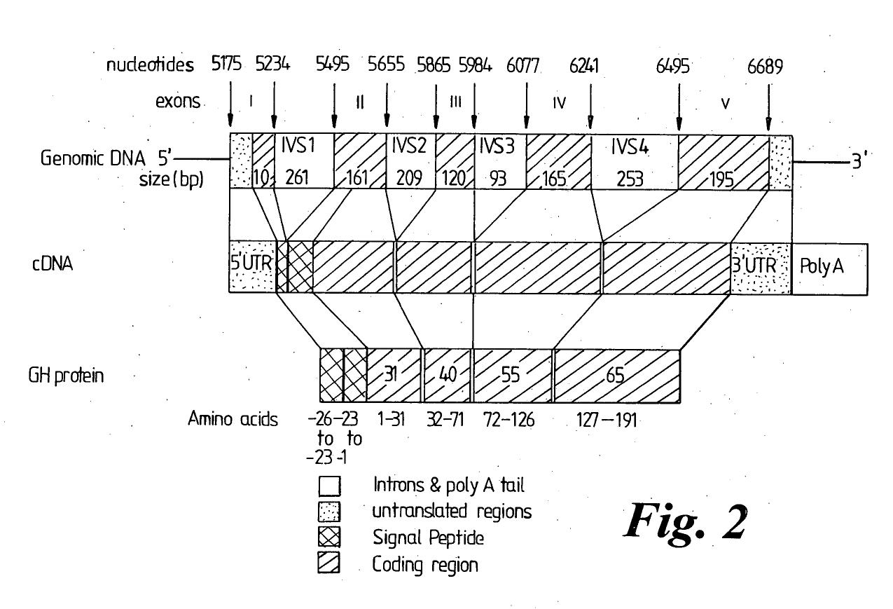

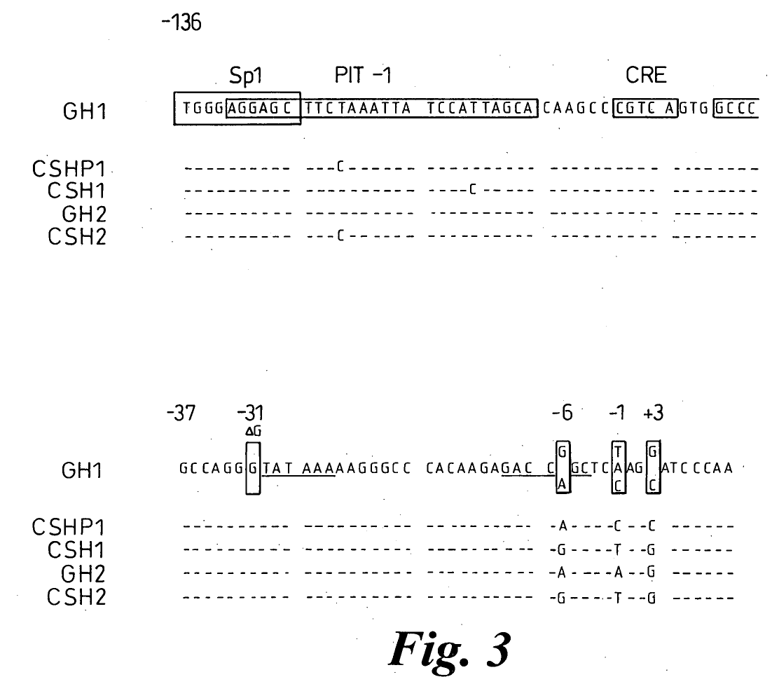

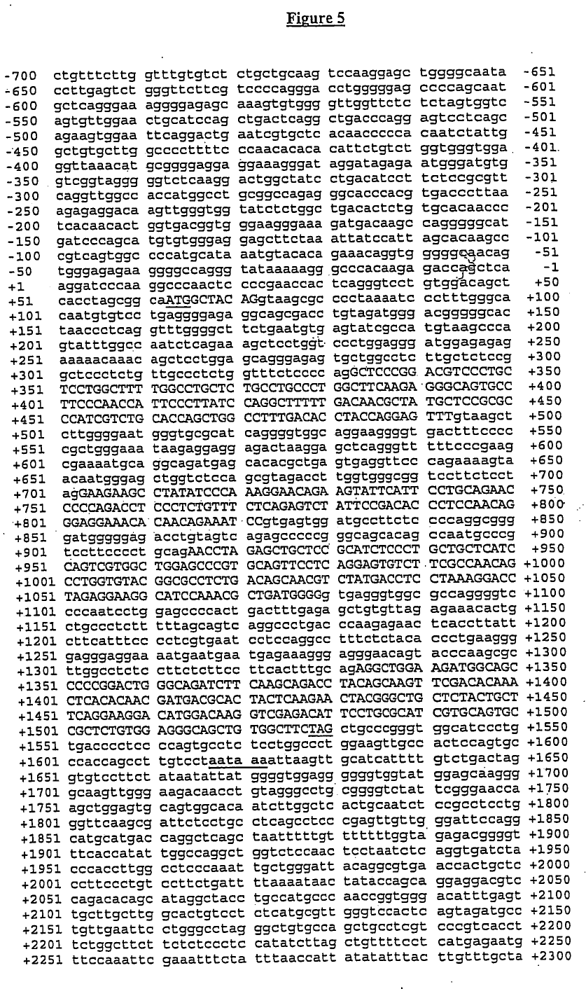

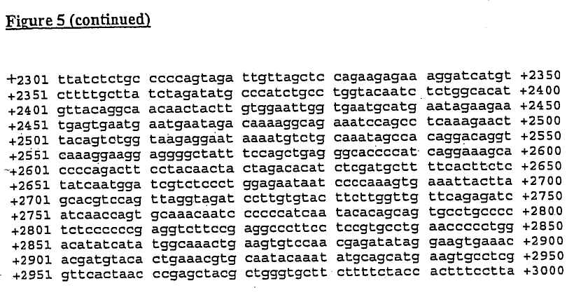

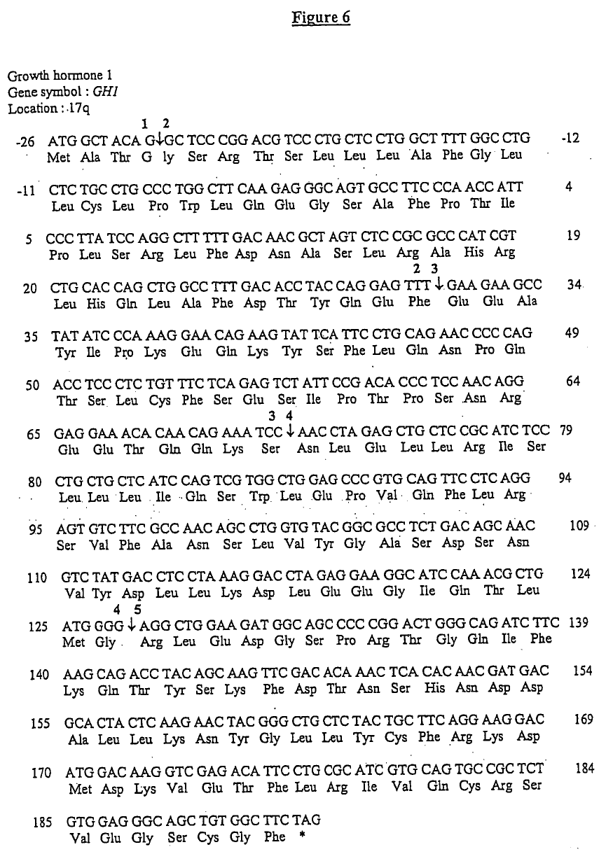

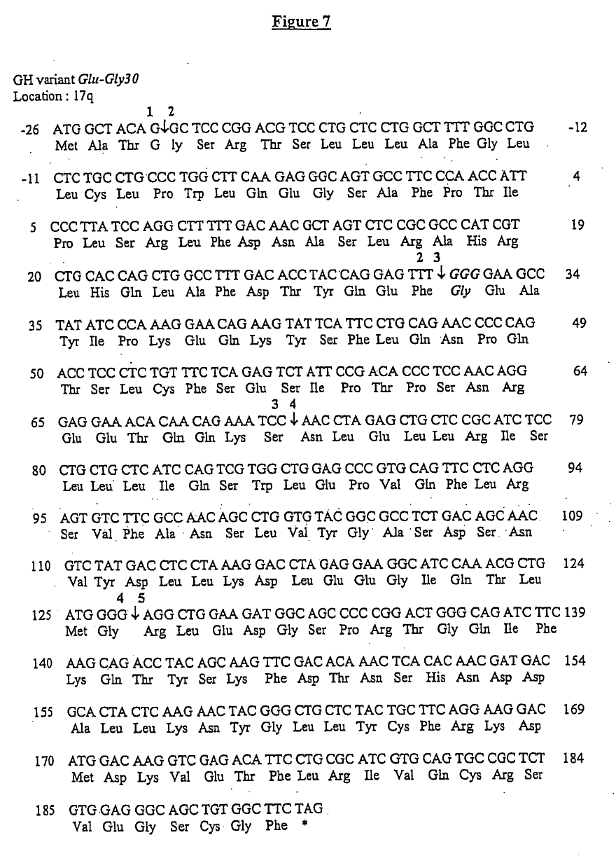

Several different GH isoforms are generated from expression of the GH1 gene (GH1 reference sequence is shown in FIG. 5). In 9% of GH1 transcripts, exon 2 is spliced to an alternative acceptor splice site 45 bp into exon 3, thereby deleting amino acid residues 32 to 46 and generating a 20 kDa isoform instead of the normal 22 kDa protein. This 20 kDa isoform appears to be capable of stimulating growth and differentiation. The factors involved in determining alternative acceptor splice site selection are not yet characterised but are clearly of a complex nature. A 17.5 kDa isoform, resulting from the absence of codons 32 to 71 encoded by exon 3, has also been detected in trace amounts in pituitary tumour tissue. Splicing products lacking either exons 3 and 4 or exons 2, 3 and 4 have been reported in pituitary tissue but these appear to encode inactive protein products. A 24 kDa glycosylated variant of GH has also been described. The amino acid sequence of the major 22 kDa isoform is presented in FIG. 6, which shows the nucleotide sequence of the GH1 gene coding region and amino acid sequence of the protein including the 26 amino acid leader peptide. Lateral numbers refer to amino acid residue numbering. Numbers in bold flanking vertical arrows specify the exon boundaries. The termination codon is marked with an asterisk. The gene encoding pituitary growth hormone (GH1) is located on chromosome 17q23 within a cluster of five related genes (FIG. 1). This 66.5 kb cluster has now been sequenced in its entirety [Chen et al. Genomics 4 479-497 (1989) and see FIG. 5]. The other loci present in the growth hormone gene cluster are two chorionic somatomammotropin genes (CSHJ and CSH2), a chorionic somatomammotropin pseudogene (CSHP1) and a growth hormone gene (GH2). These genes are separated by intergenic regions of 6 to 13 kb in length, lie in the same transcriptional orientation, are placentally expressed and are under the control of a downstream tissue-specific enhancer. The GH2 locus encodes a protein that differs from the GH1-derived growth hormone at 13 amino acid residues. All five genes share a very similar structure with five exons interrupted at identical positions by short introns, 260 bp, 209 bp, 92 bp and 253 bp in length in the case of GH1 (FIG. 2).

Exon 1 of the GH1 gene contains 60 bp of 5′ untranslated sequence (although an alternative transcriptional initiation site is present at −54), codons −26 to −24 and the first nucleotide of codon −23 corresponding to the start of the 26 amino acid leader sequence. Exon 2 encodes the rest of the leader peptide and the first 31 amino acids of mature GH. Exons 3-5 encode amino acids 32-71, 72-126 and 127-191, respectively. Exon 5 also encodes 112 bp 3′ untranslated sequence culminating in the polyadenylation site. An Alu repetitive sequence element is present 100 bp 3′ to the GH1 polyadenylation site. Although the five related genes are highly homologous throughout their 5′ flanking and coding regions, they diverge in their 3′ flanking regions.

The GH1 and GH2 genes differ with respect to their MRNA splicing patterns. As noted above, in 9% of GH1 transcripts, exon 2 is spliced to an alternative acceptor splice site 45 bp into exon 3 to generate a 20 kDa isoform instead of the normal 22 kDa. The GH2 gene is not alternatively spliced in this fashion. A third 17.5 kDa variant, which lacks the 40 amino acids encoded by exon 3 of GH1, has also been reported.

The CSH1 and CSH2 loci encode proteins of identical sequence and are 93% homologous to the GH1 sequence at the DNA level. By comparison with the CSH gene sequences, the CSHP1 pseudogene contains 25 nucleotide substitutions within its “exons” plus a G→A transition in the obligate +1 position of the donor splice site of intron 2 that partially inactivates its expression.

A number of biallelic restriction fragment length polymorphisms (RFLPs) have been reported within the GH gene region. Five of these (two BgIII, two MspI, one HincI) occur in Caucasians and Blacks whereas a further BamHI polymorphism occurs predominantly in Blacks. Strong linkage disequilibrium has been observed between these polymorphisms consistent with the relatively recent evolutionary origin of the gene cluster. The HincII and BamHI polymorphisms occur immediately 5′ to the GH1 gene.

An RsaI polymorphism occurs in the GH1 promoter region resulting from an A/G dimorphism at nucleotide −75 whilst a relatively frequent Sphl polymorphism remains to be fully characterised. A highly informative (83% heterozygosity) variable number repeat polymorphism has been located some 19 kb 3′ to the GH1 gene; formatted for PCR, the 18 distinct alleles of this polymorphism can be distinguished by fragment size (201 to 253 bp).

Finally, the GH1 gene promoter/5′-untranslated region has been found to exhibit a very high level of sequence polymorphism with 17 variant nucleotides within a 570 bp stretch (Table 2A):

TABLE 2A
Known polymorphisms in the human GH1 gene promoter/5′
untranslated region [after Giordano et al Human Genetics
100 249-255 (1997) and Wagner et al Eur. J. Endocrinol.
137 474-481]. (FIG. 3).

| Nucleotide location | Polymorphism (alternative nucleotides) |
|---|---|
| −476 | G/A |
| −364 | G/T |
| −339 | ΔG |
| −308 | T/G |
| −301 | T/G |
| −278 | T/G |
| −272 to −276 | CCAGA/SMRRR |
| −168 | T/C |
| −75 | A/G |
| −57 | G/T |
| −31 | ΔG |
| −6 | G/A |
| −1 | T/A/C |
| +3 | G/C |
| +16 | A/G |
| +26 | A/C |
| +59 | T/G |

The polymorphisms at positions −1, +3 and +59 are predicted to cause amino acid substitutions in the GHDTA protein, putatively encoded by this region of the GH1 gene promoter (see below). Some of the sequence variants occur in the same positions in which the GH1 gene differs from the other placentally-expressed genes suggesting that the mechanism might be gene conversion and that the placental genes have served as donors of the converted sequences.

In a study of prepubertal short children with GH insufficiency, Hasegawa et al [J. Clin. Endocrinol Metab 85 1290-1295 (2000)] reported an association between three polymorphisms in the GH1 gene [IVS4 C→T 1101 (also reported in Table 7A and 7B hereinbelow), T/G −278 and T/G −57] and both GH secretion and height.

Since the first GH1 gene deletions were reported, a variety of more subtle lesions have been described. In some cases, these lesions have been associated with unusual types of GH deficiency and are potentially important as a means of obtaining new insights into GH structure and function

The gene encoding growth hormone (GH1) was one of the first human genes to be cloned and the first gross gene deletions (6.7 kb type) responsible for inherited growth hormone deficiency were soon detected by Southern blotting. All gross deletions involving the GH1 gene result in severe (type IA) deficiency, characterised by the total absence of GH. About 70% of characterised deletions of the GH1 gene are 6.7 kb in length, whilst most of the remainder are of 7.6 kb or 7.0 kb (Table 2B—Gross deletions involving the GH1 gene, or in the vicinity of the GH1 gene, that cause GH deficiency and short stature).

TABLE 2B
Gross deletions involving or in the vicinity of the GH1 gene

| | | | Post-treatment |
|---|---|---|---|
| Deletion | | | antibodies |
| size (kb) | Loci involved | Comments | present? |
| 6.7 | GH1 | Swiss family | Yes |
| 6.7 | GH1 | Japanese family | Yes |
| 6.7 | GH1 | Argentinan family of | Yes |
| | | Spanish ancestry. | |
| | | Homozygous. | |
| 6.7 | GH1 | Austrian family | Yes |
| 6.7 | GH1 | Brazilian family | Yes |
| 6.7 | GH1 | Patient with short | Yes |
| | | stature and | |
| | | cystic fibrosis | |
| 6.7 | GH1 | Various | No |
| 7.6 | GH1 | Iraqi, Yemeni and | No |
| | | Iranian families | |
| 7.6 | GH1 | Italian family. | Yes |
| | | Homozygous. | |
| | | Consanguinous | |
| | | marriage | |
| 7.6 | GH1 | Italian and | Yes |
| | | Turkish families | |
| 7.6 | GH1 | Spanish family | No |
| 7.6 | GH1 | Various | Yes |
| 7.0 | GH1 | Canadian family | Yes |
| 7.0 | GH1 | Mexican family | Yes |
| 7.0 | GH1 | Chinese family. | no - No treatment |
| | | Homozygous | with GH. |
| 45 | GH1, CSHP1, | Turkish family. | Yes |
| | CSH1, GH2 | Homozygous. | |
| | | Consanguinous | |
| | | marriage | |
| 45 | GH1, CSHP1, | Italian family. | Yes |
| | CSH1, GH2 | Homozygous | |
| 45 | GH1, CSHP1, | Italian family. | Yes |
| | CSH1, GH2 | Homozygous. | |
| | | Consanguinous | |
| | | marriage | |
| 45 | GH1, CSHP1, | “Asian” family | No |
| | CSH1, GH2 | | |
| ? | CSH1, GH2, | Italian family. | No |
| | CSH2 | Heterozygous | |
| ? | CSH1, GH2, | Danish family. | No |
| | CSH2 | Compound | |
| | | heterozygous for | |
| | | non-identical | |
| | | deletions | |
| Double | (i) GH1 (6.7 kb) | French origin | Yes |
| | | (Romany). | |
| | | Homozygous. | |
| | (ii) CSH1, GH2, | Consanguinous | |
| | CSH2 (˜32 kb) | marriage. | |

In addition, several examples of much more infrequent deletions have been reported. In recent years, various attempts have been made to move away from Southern blotting toward PCR-based approaches as a mutation screening tool. Homozygous GH1 gene deletions have been fairly readily detected by PCR amplification of the GH1 gene and flanking regions followed by restriction enzyme digestion of the resulting PCR products. Although this approach has been used successfully to exclude homozygosity for a GH1 gene deletion in at-risk pregnancies, it is however unable to distinguish homozygosity for the wild-type gene from heterozygosity for a gene deletion. It would also fail to detect deletions other than the relatively short 6.7, 7.0 and 7.6 kb deletions that remove only the GH1 gene.

PCR primers have been designed which immediately flank the GH1 gene and which generate a 790 bp fragment from control DNA samples. Absence of this fragment was held to be indicative of a GH1, gene deletion but the use of “non-specific PCR fragments” as internal controls for PCR amplification must make the reliability of this method somewhat suspect.

As well as gross deletions, three micro-deletions of the GH1 gene have been reported; two of these patients were also heterozygous for the 6.7 kb GH1 gene deletion (Table 3).

TABLE 3

Micro-deletions in the GH1 gene causing GH deficiency and short
stature

| | | Codon | |
|---|---|---|---|
| | Deletion | (Numbering is | |
| | (Lower case letters denote the | relative to | Post- |
| | deleted bases. \hat{ } specifies the | translational | treatment |
| Deficiency | location of the numbered codon | initiation codon | antibodies |
| type | immediately downstream.) | ATG at −26.) | present? |
| IA | GCCTG\hat{ }CTCTGcCTGCCCTGGC | −11 | Yes |
| II | CCCCAGGCGGggatgggggagacctgtaGTC | Intron 3 (del+28 | No |
| | AGAGCCC | to +45) | |
| IA | TCTGT\hat{ }TTCTCagAGTCTATTCC | 54 | No |

Only seven different single base-pair substitutions have been reported from within the coding region of the GH1 gene (Table 4).

TABLE 4
Single base-pair substitutions in the GH1 coding
region causing GH deficiency and short stature

| | | | Codon | |
|---|---|---|---|---|
| | | | (numbering | |
| | | | relative to | |
| | | | translational | Post-treatment |
| Deficiency | Nucleotide | Amino acid | initiation codon | antibodies |
| type | substitution | substitution | ATG at −26) | present? |
| IA | ACA→GCA | Thr→Ala | −24 | No |
| IA | TGG→TAG | Trp→Term | −7 | No |
| IA | GAG→TAG | Glu→Term | −4 | Yes |
| II | CGC→TGC | Arg→Cys | 77 | No |
| ? | CCC→CTC | Pro→Leu | 89 | No |
| ? | GAC→GGC | Asp→Gly | 112 | No |
| ? | CGC→CAC | Arg→His | 183 | No |

Two of these single base-pair substitutions are nonsense mutations converting amino acid residues Trp-7 and Glu-4 in the signal peptide to stop codons. These mutations are the only known GH1 gene lesions to cause type IA deficiency that are not gene deletions. Since these lesions predict termination of translation within the signal peptide, they would be incompatible with the production of a functional GH molecule. The other five single base-pair substitutions (including ReC at codon 77, disclosed in EPA 790 305 in relation to the treatment of gigantism) are missense mutations that result in the production of dysfunctional growth hormone molecules. Such naturally-occurring mutations are very much more informative than artificially-induced mutations, in that the former can, in principle, be related directly to the clinical phenotype ie the height of the patient in question.

Single base-pair substitutions in the promoter region of possible pathological significance were first sought by sequencing the promoter region of the GH1 gene (between −60 and +70 relative to the transcriptional initiation site) in three Chinese patients with IGHD IA and 2 controls. Several differences were noted but these were probable polymorphisms and were not characterised further. As mentioned above, the promoter region of the GH1 gene has subsequently been shown to exhibit a very high level of sequence polymorphism with 17 variant nucleotides within a 570 bp stretch (FIG. 3). However, these sequence variants were not found to be over-represented in patients as compared to controls.

GH1 promoter variation has also been separately investigated and a total of 22 variant polymorphic sites were detected, mostly single base-pair substitutions: 17 of these occurred in a 550 bp region 5′ to the ATG initiation codon, three occurred around position −1075 5′ to ATG, and two occurred within intron 1 (IVS1) at positions 76 and 219 respectively [Wagner et al, Eur J Endocrinol 137 474-81 (1997)]. All except four of these variants were also noted in controls but these four variants were not considered to be the cause of the growth hormone deficiency. Only one of the variant sites occurred within a sequence homologous to a transcription factor binding site: the alternative presence of CCAGA and GAGAG sequences at −333 within a potential (but not proven) NF-1 binding site.

Therefore, to date, no mutations of pathological significance have been reported in the GH1 gene promoter.

Single base-pair substitutions affecting MRNA splicing have also been described in the GH1 gene. Most are associated with a comparatively rare dominant form of GH deficiency (Table 5).

TABLE 5
Single base-pair substitutions affecting mRNA splicing
and causing GH deficiency and short stature

| | Nucleotide | | |
|---|---|---|---|
| Deficiency | substitution/ | | Ethno-geographic |
| type | position | Splice site | origin/zygosity |
| II | G→A, +1 | IVS3 donor | Sweden, North America, |
| | | | Northern Europe, South |
| | | | Africa, Chile/heterozygous |
| II | G→C, +1 | IVS3 donor | Turkish/heterozygous |
| II | T→C, +2 | IVS3 donor | ? |
| II | G→A, +5 | IVS3 donor | Chilean/heterozygous |
| II | G→C, +5 | IVS3 donor | ? |
| II | T→C, +6 | IVS3 donor | Turkish/heterozygous |
| | | | Asian/heterozygous |
| II | G→A, +28 | IVS3 donor | ?/heterozygous |
| IB | G→C, +1 | IVS4 donor | Saudi Arabian/homozygous |
| IB | G→T, +1 | IVS4 donor | Saudi Arabian/homozygous |
| IB | G→C, +5 | IVS4 donor | ? |

The transversions in the intron 4 donor splice site have been shown by MRNA in vitro expression analysis of transfected cells to activate a cryptic splice site within exon 4, 73 bp 5′ to the exon 4 donor splice site. This would predict the generation of an aberrantly spliced product lacking amino acids 103-126 encoded by exon 4 and, as a consequence of a shift in the reading frame, the incorporation of 94 novel amino acids including 29 resulting from read-through of the normally untranslated 3′ non-coding region of the GH1 gene.

Since the region of the GH protein encoded by exons 4 and 5 is thought to be important for correct targeting of the protein to secretory granules, it has been predicted that this aberrant protein would not be secreted normally. However, no antibodies to exogenous GH have been noted in patients with type IB GH deficiency. The avoidance of immune intolerance may thus indicate that at least some of the aberrant protein product could be secreted and that it could be partially stable in the circulation. The seven known splicing mutations within IVS3 (Table 5) are associated with a type II deficiency state manifesting autosomal dominant inheritance through the affected families.

GH deficiency patients with truncating GH1 mutations or homozygous gene deletions are at considerable risk of developing anti-GH antibodies upon GH treatment. By contrast, we are not aware of any reports describing allo-antibody formation in patients with either missense mutations or single base-pair substitutions within splice sites.

Until now, no other correlations between mutant genotype and clinical phenotype have been reported. The requisite data in the published literature are sparse and very variable in quality, but we have attempted a crude meta-analysis as a means of gauging whether or not patients with gross gene deletions differ from patients with splice site mutations in terms of their clinical and phenotypic sequelae. The height of the patients with GH1 deletions was found to be on average 7.3 SD below the age-adjusted mean (n=29), as compared with an average of 5.4 SD below the mean (n=17) for the patients with GHI splicing mutations. Although bone age delay was greater and growth velocity lower in the deletion patients, such findings are very difficult to interpret since they may be subject to bias of ascertainment.

Since most cases of familial GH deficiency hitherto described are inherited as an autosomal recessive trait, some examples of the inherited deficiency state are likely to have gone unrecognized owing to small family size. Similarly, cases of GH deficiency resulting from de novo mutations of the GH1 gene could be classified as sporadic, and a genetic explanation for the disorder would neither be entertained nor sought. Finally, depending upon the criteria used for defining the deficiency state, it may be that the full breadth of both the phenotypic and genotypic spectrum of GH deficiency may never have come to clinical attention. For these reasons, current estimates of the prevalence of GH deficiency could be inaccurate and may therefore seriously underestimate the true prevalence in the population.

The definition of IGHD favoured by many combines (a) severe growth retardation, often—as mentioned above—defined as <−4.5 SD in height; (b) reduced GH response to stimulationlprovocation (ie a serum GH level of <4 ng/ml); and (c) no other cause for growth retardation. The strict adherence to formal definitions of what constitutes GH deficiency and the fairly uniform acceptance of these criteria, especially criterion (b), in selecting patients for study [Shalet SM et aL Endocrine Rev 19 203-223 (1998)] would have served to ensure that the described GH1 mutational spectrum was not only far from complete but also unrepresentative of the wider mutational spectrum. Thus, mutations responsible for GH deficiency states in which the SD scores were less severe or the GH levels less reduced (eg missense mutations within the coding region of the gene or promoter mutations) would have been much less likely to come to clinical attention. Indeed, this may go some way toward explaining why only five different missense mutations have so far been reported in the GH1 gene, a finding which is virtually unprecedented for a fairly prevalent disorder that has been studied at the molecular level for nearly 20 years (The Human Gene Mutation Database; Krawczak et al, Hum Mutation 15, 45-51 (2000)).

The complete absence of GH produces a readily recognisable and severe clinical phenotype that has been extensively studied. In those reported studies in which the phenotype of the patients is less severe and in which patient selection criteria have actually been identified, patient ascertainment strategies have generally used the deviation of an individual's height from the mean height for their age as a diagnostic indicator of growth failure.

The selection of patients using criteria (a) and (b), as defined above, will serve to define patients with a severe degree of IGHD-related growth failure. We have proposed that moderating the criteria applied in selecting patients for study would be likely to lead to the inclusion of patients whose growth failure is a manifestation of a different portion of the GH deficiency spectrum, and which could therefore yield a novel set of underlying mutational lesions. Some of these novel lesions could give rise to stable, yet dysfunctional, GH molecules that would exhibit normal immunological reactivity but little or no. biological activity. On the basis of radio-immunoassay test results, dysfunctional GH molecules would have been erroneously regarded as normal. If such dysfunctional variants were to turn out to be common, then it would follow that GH deficiency is being under-diagnosed as a result of our current dependence on radio-immunoassay-based GH “function tests”. Further, it would demonstrate an urgent need for the development of a true functional diagnostic assay.

We believe that height velocity is a more sensitive indicator of growth failure than absolute height measurements. The use of height velocity in conjunction with an assessment of bone age delay (retarded osseus maturation also due to GH deficiency), and other variables being normal, has allowed us to identify a unified group of patients with phenotypes which are less severe than that of classical IGHD patients having no GH, but who are more likely to have lesions of the GH1 gene than those selected on the basis of height measurements alone. Another important indicator is growth failure, which may or may not be accompanied by short stature and/or reduced height velocity and/or bone age delay.

Accordingly, the present invention provides a detection method for detecting a variation in GHI effective to act as an indicator of GH dysfunction in an individual, which detection method comprises the steps of:

(a) obtaining a test sample comprising a nucleotide sequence of the human GHI gene from the individual; and

(b) comparing the sequence obtained from the test sample with the standard sequence known to be that of the human GHJ gene, wherein a difference between the test sample sequence and the standard sequence indicates the presence of a variation (hereinafter “variant of GH1”) effective to act as an indicator of GH dysfunction characterised in that the test sample is obtained from an individual exhibiting the following criterion:

(i) growth failure, defined as a growth pattern [delineated by a series of height measurements; Brook CDG (Ed) Clinical Paediatric Endocrinology 3rd Ed, Chapter 9, p141 (1995, Blackwell Science)] which, when plotted on a standard height chart [Tanner et al Arch Dis Child 45 755-762 (1970)], predicts an adult height for the individual which is outside the individual's estimated target adult height range, the estimate being based upon the heights of the individual's parents.

The present invention therefore further provides a variant of GH1 detected by or detectable according to the above-described method of this invention.

The present invention also provides a transcript of a variant of GH1, such as a protein (hereinafter ‘GH variant’) comprising an amino acid sequence encoded by a variant of GH1, wherein the variant of GH1 is one detected by or detectable according to the above-described method of this invention.

(The terms ‘patient’ and ‘individual’ are used interchangeably in the context of this invention).

Useful as a reference for criterion (i) is Tanner and Whitehouse Arch Dis Child 51 170-179 (1976)]. A patient's target adult height range is calculated as the mid-parental height (MPH) with the range being the 10th to 90th centile for MPH, which is sex-dependent:

MPH if male=[father's height+(mother's height+13)]/2+ or − in the range of from 6 to 8 cm, usually 7.5 cm; and

MPH if female=[(father's height−13)+mother's height]/2+ or − in the range of from 6 to 8 cm, usually 6 cm

These are standard tests and measurements used in the field of human growth, and any other acceptable method of calculation, can be used to determine growth failure, although the above-described method based on the description in Brook (ibid, 1996) regarding the formula to apply for predicting the limits of the target height range and on the description in Tanner (ibid, 1970) regarding the standard height charts are preferred according to this invention.

This is therefore a substantially different criterion from those used hitherto in the identification of GH-dysfunctional patients, and involves prediction of the (future) adult height of a patient based on their parents' achieved height.

Preferably, in the detection method of this invention, the test sample is obtained from an individual exhibiting one or more further criteria, in addition to (i) above, namely:

(ii) height velocity below the 25^{th} centile for age; and/or

(iii) bone age delay according to the Tanner-Whitehouse scale of at least two years, when compared with chronological age; and/or

(iv) no other disorder known to cause inclusion in criteria (i) to (iii) above.

Preferably, the criteria (ii) through (iv) are applied cumulatively, so that each of (ii), (iii) and (iv) must be satisfied with respect to a particular individualpatient.

With respect to the criteria (ii) through (iv), each criterion may be assessed according to known methods and parameters readily available and described in the art, as elaborated further below:

(ii) Tanner J M, Whitehouse R H Atlas of Children's Growth (1982, London: Academic Press); and Butler et al Ann Hum Biol 17 177-198 (1990) are sources for statistics enabling a determination of the first criterion, viz that the height velocity of the patient is less than the 25^{th} centile for the patient's age.

(iii) The Tanner-Whitehouse scale for assessing years of bone age delay is described by Tanner J M, Whitehouse R H, Cameron N et al in Assessment of Skeletal Maturity and Prediction of Adult Height (1983, London: Academic Press). In the method of this invention, the individual preferably exhibits bone age delay of about 3.5 to 4 years (when compared with chronological age). Assessment of bone age delay in an individual is subject to a greater level of variation, when carried out more than once, the younger the individual, so, for example, multiple assessments of a child of age two may result in a bone age delay varying by +/−6 months, but at age 3 might vary by +/−4 months, and so on.

(iv) Since short stature may also be secondary to conditions other than GH dysfunction, test samples from patients suffering from such disorders are excluded from the method of the invention. That the patient is suffering from no other disorder that might give rise to similar symptoms to that of GH dysfunction is determined by baseline investigations. “Baseline investigations” therefore include tests to exclude, particularly, hypothyroidism; pseudo-hypoparathyroidism; malabsorption syndromes eg coeliac disease; renal and hepatic diseases; haematological disorders, such as anaemia; and a karyotype to check that a chromosome disorder such as Turner syndrome is not the cause of the growth failure. The patient may also have had a thorough clinical examination in order to exclude other causes of growth failure, for example, cardiac disease including congenital heart disease; chronic auto-immune conditions, such as rheumatoid arthritis and inflammatory bowel disease; chronic respiratory conditions, such as severe asthma or cystic fibrosis; and skeletal problems, such as achondroplasia. A full medical history will also have been taken and used to complement the medical examination in order to aid the exclusion not only of the physical disorders identified above but also of psycho-social deprivation, another well-recognised cause of growth failure in childhood.

Optionally, (v), the patient may also have been subjected to one or more growth hormone function tests. The term “growth hormone function tests” refers to tests of growth hormone secretion, such as those stimulation tests mentioned hereinbefore, particularly the insulin-induced hypoglycaemic test (IST).

GH function tests are usually carried out on patients who are short; have been clinically assessed and had their height monitored over more than one visit to an endocrine clinic; have no other detectable cause for their growth failure; and therefore warrant being subjected to an assessment of their ability to produce growth hormone secretion from their pituitary gland following an appropriate stimulus, such as the profound drop in blood glucose that results from the administration of intravenous insulin. Preferably, in the method according to this invention, the results of the individual's growth hormone function tests are normal.

In the detection method according to this invention, therefore, although current height may be measured in order to apply the above-noted criteria, this is not in itself a criterion used for selection of patients in this method. As mentioned above, prior art methods rely on standard deviation from ‘normal’ height (ie absolute growth) as the criterion for selecting patients. The present invention does not require inclusion of such criterion and therefore the present invention provides a detection method in which absolute height is or may be excluded as a selection criterion.

Increasing the breadth of the GH1 mutational spectrum will inevitably lead to a re-definition of inherited GH deficiency in molecular genetic terms. Furthermore, the recognition of novel types of short stature must eventually require the reclassification of GH deficiency as a disease entity. This will obviously have important implications for the screening and identification of individuals with short stature in whom the use of growth hormone treatment might be beneficial.

The test sample obtained from the patient in the detection method of the invention preferably comprises genomic DNA extracted from patient lymphocytes by standard procedures, such as from buccal smears, blood samples or hair. GH1 gene analysis is thereafter carried out by any suitable method for gene sequencing or polymorphism detection, including but not limited to gel or capillary electrophoresis mass spectrometry and pyrosequencing. It is preferably carried out according to the following steps:

1(a). Amplification, preferably PCR amplification, of a 3.2 kb fragment containing the GH1 gene in its entirety (promoter, five exons of the coding region, introns and untranslated regions) followed by the nested PCR of smaller, overlapping constituent fragments using primers designed so as to ensure GH1 gene specificity. As well as using six known primers, the design of novel GH1-specific primers has been found to be essential in order to avoid cross-contamination emanating from inadvertent PCR amplification of the paralogous, closely linked and highly homologous GH2, CSH1 and CSH2 genes, and the CSHP1 pseudo-gene. Accordingly, the method of the invention may comprise PCR amplification of the GH1 gene of the individual, or any individual suspected of having dysfunctional GH, using a GH1 gene-specific fragment, being a fragment unique to the GH1 gene whose sequence is not found in the four other paralogous (non-GH1) genes in the GH cluster, and one or more GH1 gene-specific primers which cannot bind to the homologous flanking regions in the four other paralogous (non-GH1) genes in the GH cluster. Preferably, the entire GH1 gene is amplified; and/or

1(b). Amplification, preferably, PCR amplification, of all or a fragment of genomic DNA spanning the Locus Control Region (hypersensitive sites I and II) approximately 15 kb upstream of the GH1 gene of the patient [Jones et al Mol Cell Biol 15 7010-21 (1995)]. The Locus Control Region (LCR) is an enhancer region that affects the level and time of GH1 transcription. The LCR is located 14 kb 5′ to the GH1 gene and is responsible for the co-ordinate expression of the genes in the GH gene cluster. PCR amplification was carried out, using novel oligonucleotide primers, on two overlapping fragments (254 bp and 258 bp) in some patients (Example 5); and a 1.9 kb LCR fragment was amplified in all patients (Example 5A); and

2. Optionally, but preferably, mutational screening of the entire GH1 gene or fragments thereof by Denaturing High Performance Liquid Chromatography (DHPLC) using the Transgenomic WAVE™ System [O'Donovan et al Genomics 52 44-49 (1998)]. This screening method was selected for use since it is extremely rapid, cheap, sensitive and reproducible and exhibits, at least in our hands, a detection efficiency >95%. “Bandshifts” detected by DHPLC would represent potential DNA sequence variants; (otherwise, direct DNA sequencing of the 3.2 kb GH1 gene-containing PCR fragment without the DHPLC step may also be employed); and

3. Characterisation of any such variants by DNA sequencing (either by automated or manual methods); and, optionally, but preferably also

4. Functional characterisation of GH1 gene lesions using methodology appropriate to the location of the lesion and the inferred mechanism of dysfunction.

Therefore, the present invention further provides novel GH1-specific primers for use in the analysis of GH1 as described above and in the examples, which primers include:

novel primers suitable for use in the DHPLC step (see Example 3, Table 6, for further details):

CTC CGC GTT CAG GTT GGC (GHD1F);
AGG TGA GCT GTC CAC AGG (GHD1R);
CTT CCA GGG ACC AGG AGC (GHD2R);
CAT GTA AGC CAA GTA TTT GGC C (GHD3F);
GGA GAA GGC ATC CAC TCA CGG (GHD4R);
TCA GAG TCT ATT CCG ACA CCC (GHD5F);
CGT AGT TCT TGA GTA GTG CGT CAT CG (GHD6R);
and
TTC AAG CAG ACC TAC AGC AAG TTC G (GHD7F);

and primers suitable for use in the LCR step (all 5′→3′), see also Examples 5 and 5A

GTGCCCCAAGCCTTTCCC (LCR15: 1159-1177);
TGTCAGATGTTCAGTTCATGG (LCR13: 1391-1412);
CCTCAAGCTGACCTCAGG (LCR25: 1346-1363);
and
GATCTTGGCCTAGGCCTCG (LCR23: 1584-1602);
and also
LCR 5A (5′ CCAAGTACCTCAGATGCAAGG 3′);
and
LCR 3.0 (5′ CCTTAGATCTTGGCCTAGGCC 3′);
and also
LCR 5.0 (5′ CCTGTCACCTGAGGATGGG 3′);
LCR 3.1 (5′ TGTGTTGCCTGGACGCTG 3′);
LCR 3.2 (5′ CAGGAGGCCTCACAAGCC 3′);
and
LCR 3.3 (5′ ATGCATCAGGGCAATCGC 3′)

are suitable for sequencing the 1.9 kb fragment.

Other novel primers, for use in PCT-amplification of the entire GH1 gene (see Example 5D) include:

| | |
|---|---|
| GH1G5 | (5′ GGTACCATGGCTACAGGTAAGCGCC 3′); |
| GH1G3 | (5′ CTCGAGCTAGAAGCCACAGCTGCCC 3′); |
| BGH3 | (5′ TAGAAGGCACAGTCGAGG 3′); |
| GH1R5 | (5′ ATGGCTACAGGCTCCCGG 3′); |
| and | |
| GH1R3 | (5′ CTAGAAGCCACAGCTGCCC 3′). |

The detection method of the invention and the variant of GH1 identifiable or detectable thereby can give rise to the following additional advantages:

1. Expansion of the known spectrum of GH1 gene mutations by identification and characterisation of new lesions.

2. Evaluation of the role of GH1 gene mutations in the aetiology of short stature.

3. Identification of the mode of inheritance of novel GH1 gene lesions.

4. Elucidation of the relationship between mutant genotype arnd clinical phenotype. This is deemed essential for the early detection and appropriate clinical management of GH deficiency.

5. Evaluation of the effects of GH1 mutations on the structure and function of the GH molecule. This is particularly important for the assessment of those children with a clinical phenotype at the milder end of the clinical spectrum of short stature. In this group of patients, dysfunctional GH may be produced that is immunologically active and therefore falls within the normal range in GH function tests.

6. Development of rapid DNA diagnostic tests for inherited GH deficiency

7. Assessment of our postulate that GH deficiency is currently under-diagnosed and underestimated in the population.

Therefore, the characterisation of further, naturally occurring GH1 lesions promises to be of considerable importance to studies of GH structure, function and expression. Studies of novel coding sequence variants should increase our understanding not only of GH function, but also of the interactions between GH and its receptor (GHR), and the process of GHR-mediated signal transduction. Insights obtained could be relevant to the rational design of a new generation of therapeutic agents. Similarly, studies of naturally-occurring GH1 lesions in the promoter region should provide new insights into the control of GH1 gene expression. Thus it may be seen that a broad spectrum of mutational lesions will necessarily improve our understanding of the relationship between mutant genotype and clinical phenotype in inherited forms of GH deficiency. Clearly, these studies are essential for the early detection and appropriate clinical management of familial GH deficiency.

The present invention therefore further provides a variant of GH1, which differs from GH1 and is detectable by the method according to the invention but is not detectable by methods used hitherto, such as those reliant on patient selection criteria based primarily on height or on other criteria or combinations thereof. Such GH1 variants of the invention include those characterised in Example 6 and especially Table 7B hereinafter. As indicated hereinbefore, current tests to assess GH secretion are many and varied and no single currently available investigation is ideal. Since the secretion of human GH is pulsatile, and because the amplitude and frequency of the GH pulses are extremely variable (being influenced by multiple internal and external factors including sleep, exercise, stress and the pubertal stage of the individual concerned), those tests that yield the best information require close supervision of the patient in a dedicated investigation ward. The tests are therefore time-consuming, expensive, and cause considerable stress and distress to the patient and their family. The insulin-induced hypoglycaemic test (IST) is of particular note; it is used by many doctors, as mentioned above, to assess GH secretion but deaths have occurred owing to the treatment necessary for the hypoglycaemia induced in the patient as a necessary requirement of its successful implementation. It is therefore of paramount importance that the decision to perform an investigation, such as an IST, is most carefully considered before it is given a place in the assessment of a short child. The development of a DNA test for use in screening short patients would therefore have many advantages over the other tests currently available.

Accordingly, the present invention provides a screening method for screening a patient suspected of having dysfunctional GH, which screening method comprises the steps of:

(a) obtaining a test sample comprising a nucleotide sequence of the human GH1 gene from the patient; and

(b) comparing a region of the sequence obtained from the test sample with the corresponding region of a predetermined sequence characterised in that the predetermined sequence is selected from a variant of GH1 detectable according to the above-described method of the present invention.

More specifically, the screening method of the invention is characterised in that the predetermined sequence is an oligonucleotide having a nucleic acid sequence corresponding to a region of a variant GH1 gene, which region incorporates at least one variation when compared with the corresponding region of the wild type sequence.

Especially preferred is when the variation is one detectable by the detection method of the invention, such as any of those identified in Example 6 and Table 7 hereinafter.

Preferably, the test sample comprises genomic DNA, which may be extracted by conventional methods.

Therefore, the present invention further provides a screening method for determining GH dysfunction, comprising:

(a) obtaining a first test sample from an individual suspected of GH dysfunction; and

(b) comparing the GH1 gene or GH1 transcript, or fragment therefrom (eg CDNA), in the first test sample to the corresponding gene, transcript or fragment of a GH1 variant obtainable from a second test sample derived from an individual exhibiting the following criterion:

(i) growth failure defined as a growth pattern [delineated by a series of height measurements; Brook CDG (Ed) Clinical Paediatric Endocrinology 3rd Ed, Chapter 9, p141 (1995, Blackwell Science)] which, when plotted on a standard height chart [Tanner et al Arch. Dis. Child 45 755-762 (1970)], predicts an adult height for the patient which is outside the patient's estimated target adult height range, the estimate being based upon the heights of the patient's parents; and/or

(ii) height velocity below the 25^{th} centile for age; and/or

(iii) bone age delay according to the Tanner-Whitehouse scale of at least two years, when compared with chronological age; and/or

(iv) no other disorder known to cause inclusion in criteria (i) to (iii) above.

Conveniently, the present invention provides a screening method for screening an individual suspected of GH dysfunction, which screening method comprises the steps of:

(a) obtaining a test sample comprising a nucleotide sequence of the human GH1 gene from an individual; and

(b) comparing a region of the sequence obtained from the test sample with the corresponding region of a predetermined sequence

wherein the predetermined sequence is selected from a GH1 variant identified or identifiable by a detection method according to this invention.

The predetermined sequence is preferably an oligonucleotide having a nucleic acid sequence corresponding to a region of a variant GH1 gene, which region incorporates at least one variation when compared with the corresponding region of the wild type sequence.

The first test sample or the test sample in the screening methods of this invention preferably comprises genomic DNA.

In the screening method of the invention, the comparison step may be carried out in conventional manner, for example by sequencing the appropriate region of the GH1 gene, particularly in the case where relatively few variants are to be detected/compared. Where relatively large numbers, of variants are involved, DNA chip technology may be employed, such as wherein the chip is a miniature parallel analytical device that is used to screen simultaneously either for multiple known mutations or for all possible mutations, by hybridisation of labelled sample DNA (cDNA or genomic DNA derived from the patient) to micro-arrays of mutation-specific oligonucleotide probes immobilised on a solid support [Southem, Trends Genet 12 110-115 (1996)].

The advantage of a DNA screening method according to the invention over current tests include:

1. It involves, for the patient, only a single blood test that can be performed in a clinic. Hospital admission, prolonged medical supervision and repeated blood sampling would not be required as is the case for the majority of currently-available tests. There would therefore be a reduction in the expense incurred, the use of specialist time and the distress caused for each patient tested.

2. Earlier diagnosis of functional GH deficiency in patients would become possible. The ease with which the DNA screen can be performed would allow the clinician to consider such an investigation much earlier in the management of a patient than might otherwise be the case. Currently, owing to the problems inherent in tests for GH secretion, doctors will assess children in the out-patient clinic over a long period of time, sometimes several years, before they will subject a child to an IST. The early diagnosis of a genetic aetiology for GH deficiency would enable earlier treatment with GH thereby bringing forward the opportunity to treat patients appropriately by months, or even years in individuals with a less severe phenotype.

3. More patients could be tested for GH dysfunction. The ease of the DNA test would allow the doctor to perform it as part of the initial assessment of all short patients at their first visit to the endocrine clinic. This is likely to reveal patients with lesions of the GH1 gene that cause severe growth problems and also those with milder lesions (e.g. missense mutations in the coding region). These patients may not previously have come to clinical attention because their clinical/phenotypic problems would not have been severe enough to warrant an IST, but they might nevertheless still benefit from treatment with GH.

4. Early identification of patients who will require life-long treatment with GH would be possible. These patients could be identified and treated appropriately without recourse to either initial testing or re-testing for GH secretion, or the use of a period without GH to assess their progress (a “trial without treatment”).

5. Easy and early identification of family members with GH dysfunction would become available. Once the genetic lesion responsible for growth problems has been identified in an individual, it is relatively easy to assess other family members for the same genetic lesion and to ascertain whether they would also gain benefit from treatment with GH.

6. Accuracy of diagnosis should increase. Tests for GH secretion are notorious for their variability in terms of reproducibility of assay results, both within and between laboratories. DNA screening would make this problem a thing of the past. In addition, GH secretion test results can be very difficult to interpret in certain situations, for example, if the patient is also hypothyroid or has delayed puberty. DNA screening would remove this doubt and prevent delay in the initiation of GH treatment for those patients in whom its use would be beneficial.

Accordingly, the present invention further provides a kit suitable for use in carrying out the screening method of the invention, which kit comprises:

(a) an oligonucteotide having a nucleic acid sequence corresponding to a region of a variant GH1 gene, which region incorporates at least one variation from the corresponding wild-type sequence; and

(b) an oligonucleotide having a nucleic acid sequence corresponding to the wild-type sequence in the region specified in (a); and, optionally,

(c) one or more reagents suitable for carrying out PCR for amplifying desired regions of the patient's DNA.

Such reagents may include, for example, PCR primers corresponding to the exon of the GH1 gene, and/or primers mentioned herein, especially novel primers mentioned hereinabove; and/or other reagents for use in PCR, such as Taq DNA polymerase.

Preferably, the oligonucleotides in the kit comprise in the range of from 20 to 25 base-pairs, such as 20 base-pairs for the variant sequences and either 20 for the wild-type in the case where the variant is a single base-pair substitution or 25 base-pairs where the variant is a 5 base-pair deletion. In any case, the oligonucleotides must be selected so as to be unique for the region selected and not repeated elsewhere in the genome.

Obviously, in the situation where it is desired to screen for multiple variations, such as in the range of from 15 to 20 or more, this would necessitate a kit comprising up to 40 oligonucleotides or more. In the alternative screening method, therefore, using DNA chip technology, the present invention provides a plurality of oligonucleotides as defined in kit component (a) above immobilised on a solid support.

Other nucleotide detection methods could be used, such as signal amplification methods being pioneered in nanotechnology (such as Q-Dots). Also, single molecule detection methods could be employed (such as STM). In which case, the kit according to this invention may comprise one or more reagents for use in such alternative methods.

Alternatively, the screening method and corresponding kit according to this invention may be based on one or more so-called ‘surrogate markers’ that are indicative of or correlated to the presence of a variant of GH1 or a GH variant, such as proteins/amino acid sequences eg antibodies specific for a GH variant or a variant of GH1. Such a “surrogate marker” may comprise:

(a) any biomolecule (including, but not limited to, nucleotides, proteins, sugars, and lipids);

(b) a chemical compound (including, but not limited to, drugs, metabolites thereof, and other chemical compounds); and/or

(c) a physical characteristic,

whose absence, presence, or quantity in an individual is measurable and correlated with the presence of a GH variant or a variant of GH1.

Further, suitable, alternative screening methods according to this invention may further comprise obtaining a test sample comprising a GH variant (ie a protein/peptide sequence comprising a variation of hGH, such as one encoded by a variant of GH1 detected by the method of this invention) that is identifiable by conventional protein sequence methods (including mass spectroscopy, micro-array analysis, pyrosequencing, etc), and/or antibody-based methods of detection (eg ELISA), and carrying out one or more such protein sequencing method(s).

In which alternative cases, the kit according to this invention may comprise one or more reagents for use in such alternative methods.

GH1 variants detectable by the detection method of this invention may have additional uses than as standards in a screening test for GH dysfunction. For example, variants other than those where the variation is in the promoter region of the GH1 gene may be used to treat a patient wherein GH production is over-stimulated, such as in cases of pituitary gigantism or acromegaly.

The present invention further provides:

(a) for the use of one or more of the GH variants or a variant of GH1 which comprises two terminating mutations for the identification of individuals who do not produce any growth hormone at all and who would be classified as classical GHD by conventional diagnostic techniques;

(b) a GH variant or a variant of GH1 which leads to modified binding of GH to the growth hormone receptor or its binding protein (ie the carrier for GH in vivo), insomuch as the transport of the variant GH from the pituitary by binding to its binding protein is impaired or inhibited leading to destruction of the unbound protein en route to the tissue receptor;

(c) a GH variant or a variant of GHI capable of disrupting the formation of the zinc dimer storage form of the GH protein in the pituitary;

(d) a GH variant or a protein expressed by a variant of GH1, being a protein with antagonist properties to the GH receptor and whose receptor binding constant determines the amount of extraneous GH (dose) needed to treat a patient in order to overcome the potency and inhibitory action of the variant protein; ie the variant protein competes with the wild type to bind to the receptor;

(e) use of the GH variant or a variant of GHI according to the invention for therapeutic, diagnostic or detection methods;

(f) use of the GH variant or a variant of GH1 according to the invention for the determination of susceptibility to a disease in an individual;

(g) use of the GH variant or a variant of GHI according to the invention for the determination of susceptibility to diabetes, obesity or infection;

(h) use of the GH variant or a variant of GHI according to the invention for determining binding defects and/or pituitary storage defects;

(i) use of the GH variant or a variant of GH1 according to the invention for the determination of the diagnostic dose of antagonist treatment in acromegaly;

(j) use of the GH variant or a variant of GH1 according to the invention for use in medical treatment;

(k) use of the variant of GHI according to the invention for use in gene therapy;

(l) use of the GH variant or a variant of GH1 according to the invention for determining one or more polymorphism(s) associated with a disease state; and

(m) use of the GH variant or a variant of GH1 according to the invention for the preparation of a therapeutic composition, diagnostics composition or kit, or detection kit.

Accordingly, the present invention further provides a composition comprising a GH variant, especially a variant detectable by the detection method of this invention and identified herein, in association with a pharmaceutically acceptable carrier therefor.

Furthermore, the invention provides:

(a) a nucleic acid sequence encoding a GH variant.

(b) a sequence substantially homologous to or that hybridises to sequence (a) under stringent conditions; or

(c) a sequence substantially homologous to or that hybridises under stringent conditions to the sequence (a) or (b) but for the degeneracy of the genetic code; or

(d) an oligonucleotide specific for any of the sequences (a), (b) or (c).

Also provided are:

(a) a vector comprising the nucleic acid sequence described above;

(b) a host cell comprising the vector (a), such as a bacterial host cell; and

(c) a process for preparing a variant of GH1, which process comprises:

(i) culturing the host cell (b); and

(ii) recovering from the culture medium the variant of GH1 thereby produced.

(d) a protein or amino acid sequence encoded or expressed by a sequence, vector, or cell as defined above in culture medium.

The present invention will now be illustrated with reference to the following Examples.

### EXAMPLE 1

### Patient Selection

Sources of Patients

Children with short stature have been identified through referral to the Regional Paediatric Growth, Endocrine and Diabetes Service at the University of Wales College of Medicine in Cardiff and by collaboration with other similar UK centres (viz Newport, Birmingham, Bristol, Wrexham, Liverpool, Stoke-on-Trent, Portsmouth and Southampton). A full clinical history has been taken including family history, pedigree, documentation of growth parameters and previously-performed endocrine investigations. Accurate auxology was recorded wherever possible for the index case, parents and siblings. Blood samples for molecular genetic analysis were taken from the index case and appropriate close relatives. Further families were referred by Professor John A. Phillips III (Nashville, Tenn., USA), Dr Mohamad Maghnie (Pavia, Italy) and Dr Tamas Niederland (Gyor, Hungary). To date, samples from 69 GH-deficient families have been collected.

Criteria Used

Criteria used for the selection of ALL patients were:

(i) Growth below lower limit of % target height range, determined as defined above per criterion (i) according to the invention;

(ii) Height velocity <25^{th} centile;

(iii) Bone age delay of at least 2, for example in the case of patient 1, 3.5-4 years when compared with chronological age;

(iv) All other investigations normal; and

(v) Growth hormone secretion tests normal.

In Table 5B: *GH FT: peak: Signifies units (IU/L) of activity in one or more standard Growth Hormone Function Tests. ‘Random’ denotes GH measurement taken randomly. ND denotes ‘test not done’. The height centile is included to demonstrate, with the data provided in Table 7B hereinbelow, that it is not an essential selection criterion to have a height substantially below the centile; we have found variations in GH/GH1 that occur even in patients not having a substantially reduced height.

TABLE 5B
Patients studied and results of criteria used

| | | Growth | Bone Age | |
|---|---|---|---|---|
| Patient | | Velocity | Delay | GH FT: peak |
| No. | Height Centile | Centile (ii) | (years) (iii) | (v) |
| 1 | <0.4 | | 3.5 | 48.6 |
| 2 | <0.4 | <25 | 2 | 20.2 at 60 |
| 3 | >50th | 25 | 1.9 | 3.7 at 60 |
| 4 | <0.4 | | | 26.7 |
| 5 | | | 3+ | |
| 6 | <0.4 | 25 | 2.8 | 28.4 at 30 |
| 7 | < & parallel to 3rd | 25 | 3 | 111.3 at 90 |
| 8 | | | | |
| 9 | | | | |
| 10 | 3rd centile | <25 | 2 | 38.7 |
| 11 | | | | |
| 12 | 0.4 | <25 | | not done |
| 13 | | | | |
| 14 | 10 to 25 | 25 | 4 | 13.2 at 60 |
| 15 | <<3 | | | 2.6 random |
| 16 | | | 2 | 4.6; normal |
| 17 | <3 | | | |
| 18 | <3 | 25 | 3.15 | |
| 19 | | | | |
| 20 | <10 | | 2 | 4.1 |
| 21 | | | | |
| 22 | | | | |
| 23 | | | | |
| 24 | | | | |
| 25 | | | | |
| 26 | 0.4 | 25 | 3 | 38.6 |
| 27 | | | | |
| 28 | <0.4 | 25 | 2 | 2.2 |
| 29 | | | | |
| 30 | | | | |
| 31 | | | | |
| 32 | | | | |
| 33a | <3 | 25 | 2.6 | |
| 33b | <3 | 25 | 1.4 | |
| 34 | | <0.4 | | 10 random |
| 35 | | | | |
| 36 | | | | |
| 37 | | | | |
| 38 | | | | |
| 39 | | | | |
| 40 | 3 to 10 | 11 | 2.6 | |
| 41a | <4 | <3 | | |
| 41b | <4 | | | 1.4 |
| 42 | | | | |
| 43 | | | | |
| 44 | | | | |
| 45 | | | | |
| 46 | | | | |
| 47 | | | | |
| 48 | | | | |
| 49 | | | | |
| 50a | | | | |
| 51 | | | | |
| 52 | | | | |
| 53 | | | | |
| 54 | | | 3.25 | |
| 55 | | | | |
| 56a | | | | |
| 56b | | | | |
| 57 | 0.4 | | | 27.3 |
| 58 | | | | |
| 59 | | | | |
| 60 | | | | |
| 61a | | | | |
| 61b | | | | |
| 62 | | | | |
| 63 | 10 | <25 | | 1.3 |
| 64 | | | | |
| 65a | | | 2 | 1 |
| 65b | | | 2 | 3 |
| 66 | <0.4 | <25 | 2 | 18.8 at 90 |
| 67 | | | | |
| 68 | | | | |
| 69 | | | | |
| 70 | <1 | 25 | | |
| 71 | | | | |
| 72a | | | | |
| 72b | <0.4 | | | |

### EXAMPLE 2

### Polymerase Chain Reaction (PCR) Amplification of a GH1-Specific Fragment

PCR amplification of a 3.2 kb GH1-specific fragment has been performed on 65 unrelated patients. Genomic DNA was extracted from patient lymphocytes by standard procedures.

Oligonucleotide primers GH1F (5′ GGGAGCCCCAGCAATGC 3′; −615 to −599) and GH1R (5′ TGTAGGAAGTCTGGGGTGC 3′; +2598 to +2616) were designed to correspond to GH1-specific sequences in order to PCR amplify a 3.2 kb single genomic DNA fragment containing the human GH1 gene using the Expand™ high fidelity system (Roche).

Two separate thin-walled 0.65 ml PCR tubes were used for each reaction. The first tube contained 500 nanograms (ng) each primer (GH1F and GH1R), 200 μM DATP, dTTP, dCTP and dGTP and 200 ng of patient genomic DNA made up to a final volume of 25 μl with sterile water. The second tube contained 5 μl 10× reaction buffer made up to a final volume of 24.25 μl with sterile water. Both tubes were placed on ice for 5 minutes. After this time, 0.75 μl of Expand™ polymerase mix was added to the second tube, the contents mixed and transferred to the first tube. The tube was centrifuged for 30 seconds and the reaction mixture overlaid with 30 μl light mineral oil (Sigma). The reaction mixture was then placed in a 480 or 9700 PCR programmable thermal cycler (Perkin Elmer) set at 95° C.

The reaction mix was then amplified under the following conditions: 95° C. for 2 minutes followed by 30 cycles of 95° C. for 30 seconds, 58° C. for 30 seconds and 68° C. for 2 minutes. For the last 20 cycles, the elongation step at 68° C. was increased by 5 seconds per cycle. This was followed by a further incubation at 68° C. for 7 minutes and the reaction was then cooled to 4° C. prior to further analysis. For each set of reactions, a blank (negative control) was also set up. The blank reaction contained all reagents apart from genomic DNA and was used to ensure that none of the reagents were contaminated. A one-tenth volume (5 μl) was analysed on a 1.5% agarose gel to assess whether PCR amplification had been successful before nested PCR was performed. Those samples that had PCR-amplified successfully were then diluted 1 in 100 prior to use for nested PCR.

### EXAMPLE 3

### Nested-PCR

Nested PCR was performed on the fragments produced in Example 2 to generate, in each case, seven overlapping sub-fragments that together span the entire GH1 gene. In addition, the Locus Control Region has been PCR-amplified (see-Example 5) in all but three patients.

The seven overlapping sub-fragments of the initial 3.2 kb PCR product were PCR-amplified using Taq Gold DNA polymerase (Perkin-Elmer). Oligonucleotides used for these reactions are listed in Table 6 together with their sequence locations as determined from the GH1 gene reference sequence.

A 1 μl aliquot of the diluted long (3.2 kb) PCR product was put into a thin-walled 0.2 ml PCR tube or into one well of a 96-well microtitre plate. To this was added 5 μl 10× reaction buffer, 500 ng appropriate primer pair (e.g. GHIDF and GHIDR), dATP, dTTP, dCTP and dGTP to a final concentration of 200 μM, sterile water to a volume of 49.8 μl, followed by 0.2 μl Taq Gold polymerase.

The tube or microtitre plate was then placed in a Primus 96 thermal cycler (MWG Biotech) and cycled as follows: 12 min 95° C. followed by 32 cycles of 95° C. for 30 seconds, 58° C. for 30 seconds and 72° C. for 2 minutes. This was followed by further incubation at 72° C. for 10 minutes and the reaction was then cooled to 4° C. prior to further analysis.

A one-tenth volume (5 μl) of the reaction mix was analysed on a 0.8% agarose gel to determine that the reaction had worked before denaturing high-pressure liquid chromatography (DHPLC) was performed on a WAVE™ DNA fragment analysis system (Transgenomic Inc. Crewe, Cheshire, UK). To enhance heteroduplex formation, the PCR product was denatured at 95° C. for 5 minutes, followed by gradual re-annealing to 50° C. over 45 minutes. Products were loaded on a DNAsep column (Transgenomic Inc.) and eluted with a linear acetonitrile (BDH Merck) gradient of 2%/min in a 0.1M triethylamine acetate buffer (TEAA pH 7.0), at a constant flow rate of 0.9 ml/minute. The start and end points of the gradient were adjusted according to the size of the PCR product. Analysis took 6.5-8.5 minutes per amplified sample, including the time required for column regeneration and equilibration. Samples were analysed at the Melt temperatures (TM) determined using the DHPLCMelt software (http://insertion.stanford.edu/melt.html) and listed in Table 6. Eluted DNA fragments were detected by an UV-C detector (Transgenomic Inc.).

TABLE 6

Oligonucleotide primers used for DHPLC analysis and DNA
sequencing

| | | | | DHPLC melt |
|---|---|---|---|---|
| Fragment | Primer | Sequence (5′ to 3′) | Position | temperature |
| 1 | GH1DF | CTCCGCGTTCAGGTTGGC | −309 to −292 | 60° C. |
| | GH1DR | CTTGGGATCCTTGAGCTGG | −8 to +11 | |
| 2 | GH2DF | GGGCAACAGTGGGAGAGAAG | −59 to −40 | 63° C. |
| | GH2DR | CCTCCAGGGACCAGGAGC | +222 to +239 | |
| 3 | GH3DF | CATGTAAGCCCAGTATTTGGCC | +189 to +210 | 62° C. |
| | GH3DR | CTGAGCTCCTTAGTCTCCTCCTCT | +563 to +586 | |
| 4 | GH4DF | GACTTTCCGCCGCTGGGAAA | +541 to +560 | 62° C. |
| | GH4DR | GGAGAAGGCATCCACTCACGG | +821 to +841 | |
| 5 | GH5DF | TCAGAGTCTATTCCGACACCC | +772 to +792 | 62° C. |
| | GH5DR | GTGTTTCTCTAACACAGCTCTC | +1127 to +1148 | |
| 6 | GH6DF | TCCCCAATCCTGGAGCCCCACTGA | +1099 to +1122 | 62° C. |
| | GH6DR | CGTAGTTCTTGAGTAGTGCGTCATCG | +1410 to +1435 | |
| 7 | GH7DF | TTCAAGCAGACCTACAGCAAGTTCG | +1369 to +1393 | 57° C. |
| | | | | and |
| | | | | 62° C. |
| | GH7DR | CTTGGTTCCCGAATAGACCCCG | +1731 to +1752 | |

### Example 4

### Cloning and DNA-Sequencing of GH1-Specific Long PCR Fragments

Cloning

DHPLC analysis allowed the identification of DNA fragments containing putative DNA sequence changes. To determine which allele possessed the putative sequence change, GH1-specific long (3.2 kb) PCR fragments were cloned into the PCR plasmid cloning vector pGEM-T (Promega). Cloning was accomplished by adding 50 ng of GH1-specific long PCR fragment to 10 ng pGEM-T in the presence of 1× reaction buffer and 1 μl (3 units) T4 DNA ligase in a final volume of 10 μl. The reactions were incubated for 16 hours at 10° C. The entire reaction mixture was placed in a 1.5 ml tube and cooled on ice. 50 μl DH5α competent cells (Life Technologies) were added and the tube left on ice for 30 minutes. The mixture was then heat-shocked for 20 seconds at 37° C. and returned to ice for 2 minutes. After this time, 0.95 ml of YT×2 medium (16 g tryptone, 10 g yeast extract, 5 g NaCl per litre water) was added and the mixture incubated at 37° C. for one hour with shaking. The mixture was then plated out onto pre-warmed agar plates containing 50 μg/ml ampicillin, IPTG and X-gal and incubated at 37° C. for 16 hours to allow single colonies to grow.

Eight white colonies from each plate were picked and transferred to a second gridded plate. A small amount of each bacterial colony was PCR-amplified using primers GH1DF and GH1DR (see Example 3, Table 6) and the conditions previously described to determine that the GH1-specific long PCR fragment had been successfully cloned.

Clones that contained the GH1-specific long PCR fragment were grown in 2 ml YT×2 medium; plasmid DNA was extracted from the bacteria using a Qiagen spin miniprep kit according to the manufacturer's instructions. DNA extracted in this way was quantified by measuring its optical density at 260 nm and electrophoresed on a 0.8% agarose gel to verify that the size of the clone was correct. Four of these clones were then sequenced.

Automated DNA sequencing

Clones containing the GH1-specific long PCR fragment were sequenced with the BigDye sequencing kit (Perkin Elmer) in either 0.2 ml tubes or 96-well microtitre plates in a Primus 96 (MWG) or 9700 (Perkin Elmer) PCR thermal cycler. Oligonucleotide primers used for sequencing were:

GH1S1
(5′ GTGGTCAGTGTTGGAACTGC 3′: −556 to −537);
GH3DF
(5′ CATGTAAGCCAAGTATTTGGCC 3′: +189 to +210);
GH4DF
(5′ GACTTTCCCCCGCTGTAAATAAG 3′: +541 to +560):
and
GH6DF
(5′ TCCCCAATCCTGGAGCCCCAGTGA 3′: +1099 to +1122).

1 μg of cloned DNA was sequenced with 3.2 pmol of the appropriate primer and 4[11 BigDye sequencing mix in a final volume of 20 μl. The tube or microtitre plate was then placed in the thermal cycler and cycled as follows: 2 minutes 96° C. followed by 30 cycles of 96° C. for 30 seconds, 50° C. for 15 seconds and 60° C. for 4 minutes. The reaction was then cooled to 4° C. prior to purification.

Purification was performed by adding 80 μl 75% isopropanol to the completed sequencing reaction. This was then mixed and left at room temperature for 30 minutes. The reaction was then centrifuged at 14,000 rpm for 20 minutes at room temperature. The supematant was then removed and 250 μl 75% isopropanol was added to the precipitate. The sample was mixed and centrifuged for 5 minutes at 14,000 rpm at room temperature. The supernatant was removed and the pellet dried at 75° C. for 2 minutes.

Samples were then analysed on an ABI Prism 377 or 3100 DNA sequencer.

### Example 5

### Analysis of the Growth Hormone Locus Control Region

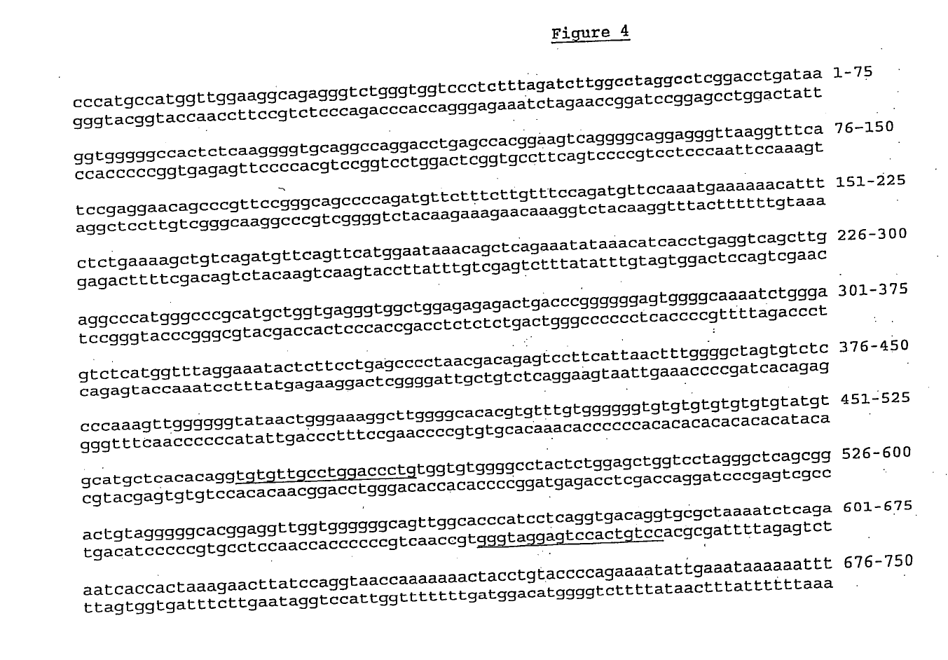

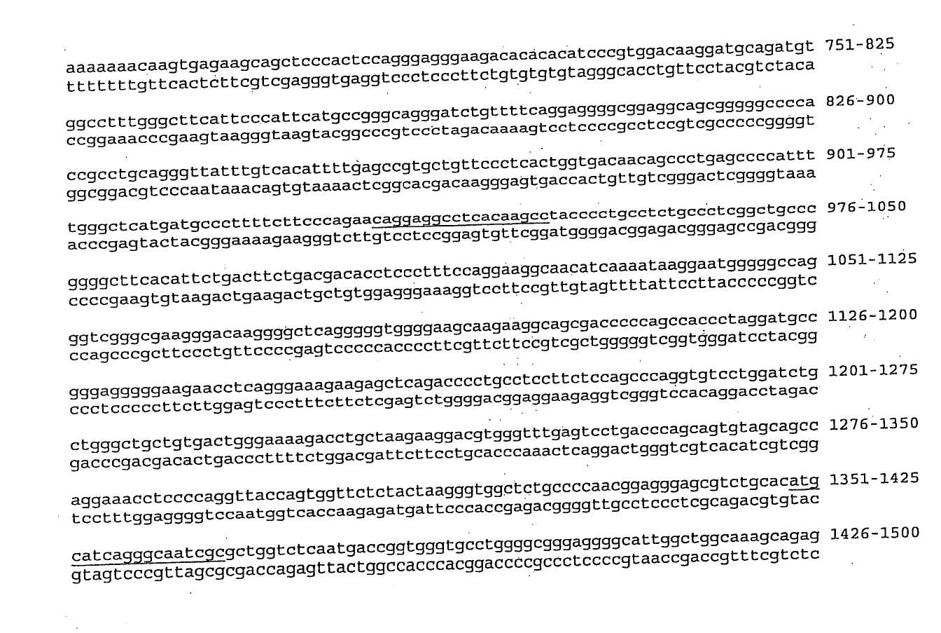

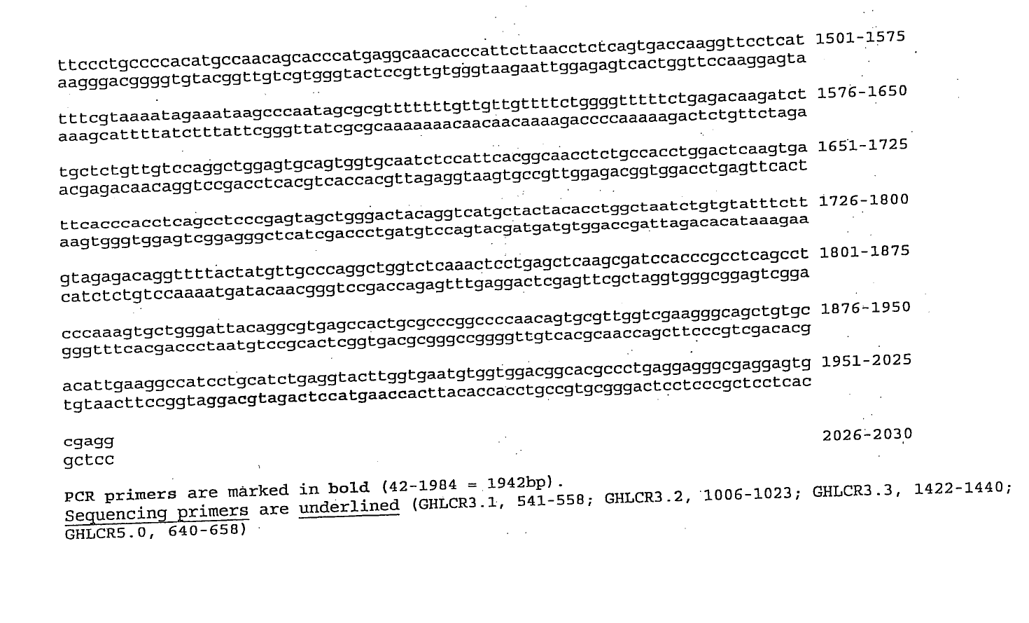

A DNA region approximately 14.5 kb upstream of the human GH1 gene is known to be involved in the tissue-specific and developmental control of GH1 gene transcription [Jin et al Mol Endocrinol 13 1249-1266 (1999)]. This is known as the Locus Control Region (LCR) and its DNA sequence was obtained from GenBank (Accession Number: AF010280). Nucleotide numbering is based on the GH LCR reference sequence (FIG. 4).

The polymorphic site at position 1192 is marked in bold type and underlined. Part of this region was analysed by PCR and DHPLC.

Two overlapping PCR fragments spanning approximately 400 bp were generated through the use of novel oligonucleotide primers designed by reference to the available DNA sequence:

Fragment 1 primers were
LCR15 (5′ GTGCCCCAAGCCTTTCCC 3′: 1159-1177)
and
LCR13 (5′ TGTCAGATGTTCAGTTCATGG 3′: 1391-1412);
and
fragment 2 primers were
LCR25 (5′ CCTCAAGCTGACCTGAGG 3′: 1346-1363)
and
LCR23 (5′ GATCTTGGCCTAGGCCTCG 3′: 1584-1602).

PCR was performed using Taq Gold polymerase: 1 μl patient genomic DNA was placed into a thin walled 0.2 ml PCR tube or into one well of a 96-well micotitre plate. To this was added, 5 μl 10× reaction buffer, 500ng of the appropriate primer pair (e.g. GH1DF and GH1DR), dATP, dTT-P, dCTP and dGTP to a final concentration of 200 μM, sterile water to a volume of 49.81 μl followed by 0.2 μl Taq Gold polymerase. The tube or microtitre plate was then placed in a Primus 96 thermal cycler (MWG Biotech) and cycled as follows: 12 minutes 95° C. followed by 32 cycles of 95° C. for 30 seconds, 58° C. for 30 seconds and 72° C. for 2 minutes. This was followed by a further incubation at 72° C. for 10 minutes and the reaction was then cooled to 4° C. prior to further analysis.

A one-tenth volume (5 μl) was analysed on a 1.5% agarose gel to determine that the reaction had worked before denaturing high-pressure liquid chromatography (DHPLC) was performed. Analysis by DHPLC was performed as described in Example 3 with a melt temperature of 61° C.

### Example 5A

### Further Analysis of the Growth Hormone Locus Control Relion

600 ng DNA from 40 control individuals and 40 patients with inherited GH deficiency were used to PCR-amplify a 1.9 kb LCR fragment using the following novel primers:

| | |
|---|---|
| LCR 5A | (5′ CCAAGTACCTCAGATGCAAGG 3′); |
| LCR 3.0 | (5′ CCTTAGATCTTGGCCTAGGCC 3′; |

and

see FIG. 4), 5 nmM dNTPs and Roche High Fidelity DNA polymerase. Reaction conditions were 98° C.×2 min, 94° C.×15s, 58° C.×30s, 72° C.×1 min×10 cycles, 58° C.×30s, 72° C.×1 min+5 seconds added on to each successive cycle×20 cycles. PCR reaction products were separated on a 2% agarose gel and bands corresponding to the LCR fragment excised with a scalpel. Agarose was removed by gel extraction and DNA eluted for sequencing. The 1.9 kb LCR fragment was sequenced on an ABI 3100 automated sequencer using the following novel primers:

| | |
|---|---|
| LCR 5.0 | (5′ CCTGTCACCTGAGGATGGG 3′); |
| LCR 3.1 | (5′ TGTGTTGCCTGGACCCTG 3′); |
| LCR 3.2 | (5′ CAGGAGGCCTCACAAGCC 3′); |
| and | |
| LCR 3.3 | (5′ ATGCATCAGGGCAATCGC 3′) |

were used to span the region.

### Example 5B

### Characterization of GH1 Promoter Haplotypes and Putative Promoter Mutations by Luciferase Reporter Gene Assay

The QuikChange™ site-directed mutagenesis kit was used to incorporate specific sequence variants into the pGL3-GH1 construct. The strategy involved annealing two complementary oligonucleotide primers, each containing the desired mutation, to opposite strands of the wild-type construct. The primers were then extended by the high fidelity Pfu DNA polymerase, resulting in a high specific mutation efficiency with a low level of random mutations. Finally, the parental DNA, which was dam methylated,was digested with DpnI, a restriction enzyme specific for methylated or hemi-methylated DNA, to select for mutation-containing plasmids.

Liposome-mediated transfection was chosen for DNA transfer into rat GH3 and human HeLa cells owing to its simplicity and efficiency. The reagent used for the transient transfection of the GH3 cells was Tfx™-50. This contained a mixture consisting of synthetic cationic lipid molecule (N,N,N′,N′-tetramethyl-N,N′-bis(2-hydroxyethyl)-2,3-di(oleoyloxy)-1,4-butanediammonium iodide) and L-dioleoyl phosphatidylethanolamine (DOPE). On hydration with water, these lipids form multilamellar vesicles, which associate with nucleic acids and facilitate their transfer into cells. Cells were plated out using a 96 well plate format. Confluent cells were removed from culture flasks, diluted with fresh medium and calculated to a cell density of 160% confluence per well. A volume of 200 μl of diluted cells was aliquoted into each well and the plate incubated at 37° C. in the presence of boxes containing moistened paper overnight. This resulted in the cells being approximately 80% confluent when transfected the following day.

The transfection mixture contained serum-free medium, DNA (pGL3-GH1 and pRL-CMV) and Tfx™-50 Reagent. A total volume of 90 μl per well was prepared containing 0.25 μg of pGL3 construct, 2 ng of pRL-CMV, and 0.5 μl of Tfx™-50 Reagent (this provided the optimised 3:1 ratio of Tfx™-50 Reagent to DNA required). The medium and DNA were mixed first, followed by the Tfx™-50 Reagent. The solution was vortexed immediately and incubated for 20 minutes at room temperature. At the 15 minute stage, the cultured wells were taken from the incubator and the growth medium removed. The Tfx™-50 Reagent/DNA mixture was briefly vortexed before 90 μl was added to each well. The plates were replaced in the incubator for 1 hour before 200 μl of pre-warmed (37° C.) complete medium was added to each well. The cells were replaced in the incubator for a further 24 hours before being lysed for the reporter assay. Transfection of HeLa cells was essentially the same as for the GH3 cells. The difference was that Tfx™-20 was used instead of Tfx™-50, 1 ng of pRL-CMV was co-transfected and the cells were calculated to a cell density of 60% confluence per well.

Cultured, transfected cells were taken from the 37° C. incubator and the growth medium removed before the addition of 50 μl of phosphate buffered saline (PBS). The plate was gently swirled before the rinse solution was removed. A 20 μl volume of passive lysis buffer was added to each culture well, ensuring the cell monolayer was completely covered. The plate was placed on a rotating table and left at room temperature for 30 mins before being stored at −70° C. The plate was thawed and spun at 6000 rpm for 20 seconds. A microplate luminometer was programmed to perform a 2 second pre-measurement delay followed by a 10 second measurement period for each reporter assay. A 50 μl volume of luciferase assay reagent II (from the Dual Luciferase Reporter Assay System (from Promega, UK)) was directly injected into the first well and the firefly luciferase activity was measured and recorded. A 50 μl volume of Stop & Glo™ reagent was then injected and the Renilla luciferase activity was recorded. This procedure was repeated for each cell lysate.

### Example 5C

### Assay of Signal Transduction Activity of GH Variants

A HK293 cell clone was selected as the target for the GH variants to be studied in our bioassay, since these cells exhibit elevated expression of the GH receptor. Prior to the assay, the cells were placed into 24-well plates (100,000 cells per well) for 24 hours, then co-transfected with a STAT 5-responsive luciferase reporter gene construct and a constitutively expressed R-Gal plasmid (CMV promoter) to allow correction for transfection efficiency. After an overnight transfection, the cells were washed and incubated with variant and wild-type GH diluted to a known standard range of concentrations for 6 hours. During this period, activation of the GH receptor would cause STAT 5 activation and luciferase expression. Thus, expression of luciferase in the assay provides a measure of the degree of GH receptor activation ie the biological activity of the GH applied to the cells. After the 6 hour incubation period, the cells were lysed and the luciferase measured in a plate reading luminometer using standard methods (assay according to the method of Ross R J M et al in Molec Endocrin 11 265-73 (1997); kit supplied by Promega UK Ltd).

### Example 5D

### In Vitro Splicing Assay

The entire human GH1 gene was PCR-amplified using the novel oligonucleotide primers:

| | |
|---|---|
| GH1G5 | (5′ GGTACCATGGGTACAGGTAAGCGCC 3′); |
| and | |
| GH1G3 (5′ CTCGAGCTAGAAGCCACAGCTGCCC 3′) | |

to amplify a 1467 bp fragment which possessed the restriction enzyme recognition sites for either KpnI(GH1G5) or XhoI(GH1G3) added to the 5′ end of the appropriate primer. These sites are underlined. PCR amplification conditions were as follows: 10 cycles 95° C. 45 sec, 58° C. 45 sec, 68° C. 2 min followed by 20 cycles 95° C. 45 sec, 68° C. 2 min plus 5 secs every cycle.

The amplified fragment was then digested with the restriction enzymes KpnI and XhoI and cloned into the plasmid vector pCDNA3.1 (Invitrogen) which had been digested with the same restriction enzymes. Once cloned, the fragments were sequenced to check for errors. The recombinant plasmid was then transfected into rat anterior pituitary GH3 cells. Following transfection, cells were left for 24 hrs. RNA was then extracted using RNAzol B (Biogenesis).

This RNA was then used for reverse transcription using the novel primer:

BGH3 (5′ TAGAAGGCACAGTCGAGG 3′)

and Superscript II (Life Technologies). 5 μg total RNA was added to 500 ng BGH3 in a final volume of 121 μl and heated to 70° C. for 15 min. The sample was then chilled on ice followed by the addition of 41 μl 5× buffer, 2 μl 0.1M DTT and 1 μl 10 nmM dNTP's. The sample was heated to 42° C., 200U (1 μl) Superscript II added, and the sample left for 50 min at this temperature. The Superscript II was then inactivated by heating to 70° C. for 15 min.

This reverse-transcribed RNA was then used for PCR. 4 μl reverse transcription mix was used in the PCR reaction with novel oligonucleotide primers:

| | |
|---|---|
| GH1R5 | (5′ ATGGCTACAGGCTCCCGG 3′); |
| and | |
| GH1R3 | (5′ CTAGAAGCCACAGCTGCCC 3′) |

to amplify a fragment of 654 bp using the following PCR cycle: 10 cycles 95° C. 45 sec, 58° C. 45 sec, 68° C. 2 min followed by 20 cycles 95° C. 45 sec, 58° C. 45 sec, 68° C. 2 min plus 5 sec every cycle. PCR products were then electrophoresed on a 1.5% agarose gel, purified and sequenced.

### Example 6

### GH1 Gene Mutations and Polymorihisms

The selection characteristics according to the present invention have, to date, led to the characterisation and identification of some 54 different and novel variants (“mutations”—Table 7B) in the GH1 gene that, on the basis of different types of evidence presented below, may be involved in the aetiology of short stature. These novel lesions comprise 31 different missense mutations, 21 different mutations in the promoter/5′-untranslated region and 2 splice site mutations. In addition, we have detected 71 polymorphisms within the GH1 gene region (Table 7A).

TABLE 7A
Polymorphisms found in the human GH1 genes of
patients (introns, coding sequence and 3′ UTR).
The nucleotides at the analogous positions of the
paralogous GH2, CSH1, CSH2 genes and the
CSHP pseudogene are given for comparison.

| | Nucleotide | Change | GH1 | GH2 | CSH1 | CSH2 | CSHP |
|---|---|---|---|---|---|---|---|
| IVS1 | 124 | A → G | A | G | A | A | G |
| IVS1 | 128 | A → T | A | T | C | C | C |
| IVS1 | 134 | A → G | A | A | A | A | A |
| IVS1 | 135 | G → T | G | G | G | G | G |
| IVS1 | 135 | G → C | G | G | G | G | G |
| IVS1 | 136 | A → G | A | A | A | A | A |
| IVS1 | 141 | A → G | A | A | A | A | A |
| IVS1 | 179 | T → C | T | T | T | T | T |
| IVS1 | 188 | C → T | C | C | C | C | C |
| IVS1 | 218 | G → A | G | G | G | G | G |
| IVS1 | 226 | C → G | C | C | C | C | C |
| IVS1 | 230 | T → C | T | T | T | T | T |
| IVS1 | 234 | T → C | T | T | T | T | T |
| IVS1 | 236 | G → C | G | G | G | G | G |
| IVS1 | 249 | A → G | A | A | A | A | A |
| IVS1 | 281 | T → C | T | C | C | C | T |
| IVS1 | 284 | T → A | T | T | T | T | T |
| IVS1 | 284 | T → C | T | T | T | T | T |
| IVS1 | 286 | G → C | G | G | G | G | G |
| IVS1 | 303 | T → C | T | T | T | T | T |
| IVS1 | 313 | G → A | G | G | G | G | G |
| IVS2 | 508 | delA | A | A | A | A | A |
| IVS2 | 519 | A → T | A | T | G | G | G |
| IVS2 | 524 | G → A | G | A | G | G | G |
| IVS2 | 558 | A → G | A | A | A | A | A |
| IVS2 | 565 | A → G | A | G | G | G | G |
| IVS2 | 573 | A → G | A | A | A | A | A |
| IVS2 | 580 | A → G | A | A | A | A | A |
| IVS2 | 585 | A → G | A | A | A | A | A |
| IVS2 | 620 | G → A | G | G | G | G | G |
| IVS2 | 622 | A → G | A | A | A | A | A |
| IVS2 | 649 | T → C | T | T | C | C | T |
| IVS2 | 665 | T → C | T | T | T | T | T |
| IVS2 | 670 | A → G | A | A | A | A | A |
| IVS2 | 676 | G → A | G | G | G | G | G |
| IVS2 | 685 | G → A | G | G | G | G | G |
| IVS3 | 836 | T → C | T | T | G | G | G |
| IVS3 | 839 | T → C | T | T | T | T | T |
| IVS3 | 879 | C → G | C | C | C | C | C |
| IVS3 | 883 | C → A | C | C | C | C | C |
| IVS3 | 901 | T → C | T | T | T | T | T |
| exon 4 | 1010 | C → T | C | T | T | T | T |
| IVS4 | 1097 | G → A | G | G | G | G | G |
| *IVS4 | 1101 | C → T | C | C | A | G | C |
| IVS4 | 1114 | C → T | C | C | C | C | C |
| IVS4 | 1169 | T → A | T | T | T | T | T |
| IVS4 | 1182 | C → T | C | C | C | C | C |
| IVS4 | 1189 | A → G | A | A | A | A | A |
| IVS4 | 1193 | A → G | A | A | A | A | A |
| IVS4 | 1196 | T → G | T | G | T | T | A |
| IVS4 | 1196 | T → C | T | G | T | T | A |
| IVS4 | 1208 | T → C | T | T | T | T | T |
| IVS4 | 1212 | C → T | C | C | C | C | C |
| IVS4 | 1216 | T → G | T | T | T | T | T |
| IVS4 | 1219 | A → G | A | A | A | A | A |
| IVS4 | 1232 | T → C | T | T | T | T | T |
| IVS4 | 1240 | A → G | A | A | A | A | A |
| IVS4 | 1243 | C → T | C | C | C | C | C |
| IVS4 | 1261 | A → G | A | A | A | A | A |
| IVS4 | 1274 | G → T | G | G | G | G | G |
| IVS4 | 1302 | T → G | T | T | T | T | T |
| exon 5 | 1341 | A → G | A | A | A | A | A |
| exon 5 | 1347 | C → T | C | C | C | C | C |
| exon 5 | 1410 | C → T | C | C | C | C | C |
| 3′UTR | 1536 | C → T | C | C | C | C | C |
| 3′UTR | 1558 | T → C | T | T | T | T | T |
| 3′UTR | 1607 | G → A | G | G | G | G | G |
| 3′UTR | 1630 | T → C | T | T | T | T | T |
| 3′UTR | 1648 | T → C | T | T | T | T | T |
| 3′UTR | 1654 | T → C | T | T | T | T | T |
| 3′UTR | 1659 | C → T | C | C | C | C | C |

*IVS4 1101 is known from Hasegawa, ibid.

In Table 7B, nucleotide numbering is based on the GH1 reference sequence shown in FIG. 5, in which the five exons of the human GH1 coding sequence are shown in upper case; the translation initiation (ATG) and termination codons (TAG) are underlined; the poly(adenylation) signal is shown in bold and is underlined; the 3′ UTR boundary is at position +1642; and +1=transcriptional initiation site. All numbering of mutational lesions, polymorphisms and oligonucleotide primers referred to in the text (with the exception of the Locus Control Region; see FIG. 4) can be related to the GH1 reference sequence.

TABLE 7B
Growth hormone deficiency: GH1 gene mutations and polymorphisms

| | | Promoter haplotype^{b} | | | | | | | | | | | | |
|---|---|---|---|---|---|---|---|---|---|---|---|---|---|---|
| | | −168 | −75 | −57 | −31 | −6 | −1 | +3 | +16 | +26 | +59 | | | |
| Patient^{a} | | (T/C) | (A/G) | (G/T) | (ΔG) | (G/A) | (T/A/C) | (G/C) | (A/G) | (A/C) | (T/G) | Mutation^{b,c,d} | Reference | Polymorphisms^{b,c,d} |
| 1 | (3) | T | A | T | G | A | A | G | A | A | T | ND | | ND |
| 1 | (6) | T | A | G | G | G | T | G | A | A | T | Arg64Gly | Unpublished | IVS4 T → A 1169 |
| | | | | | | | | | | | | (AGG → GGG: 799) | | |
| 2 | (2) | T | A | T | G | A | A | G | A | A | T | ND | | IVS4 C → T 1114 |
| 2 | (11) | T | A | G | — | G | A | G | A | A | T | ND | | IVS4 T → A 1169 |
| | | | | | | | | | | | | | | IVS4 G → T 1274 |
| 3 | (5) | T | A | T | G | A | A | G | A | A | T | ND | | ND |
| 3 | (6) | T | A | G | G | A | A | G | A | A | T | Leu163Pro | Unpublished | IVS1 A → G 141 |
| | | | | | | | | | | | | (CTC → CCC: 1442) | | |
| 4 | (1) | T | A | T | G | A | A | G | A | A | T | ND | | IVS2 A → G 622 |
| | | | | | | | | | | | | | | IVS4 T → C 1196 |
| 5 | (2) | T | A | G | G | A | A | G | A | A | T | ND | | IVS2 A → G 565 |
| 5 | (4) | T | A | G | — | A | A | G | A | A | T | ND | | IVS1 A → G 134 |
| | | | | | | | | | | | | | | IVS1 T → C 179 |
| 6 | (1) | T | A | G | — | G | A | G | A | A | T | Acceptor splice | Unpublished | IVS4 T → A 1169 |
| | | | | | | | | | | | | site IVS2 | | |
| | | | | | | | | | | | | G → A | | |
| | | | | | | | | | | | | at −1 (702) | | |
| 6 | (3d) | T | A | G | G | G | A | G | A | A | T | A → G −9 | | IVS4 T → A 1169 |
| 6 | (8d) | T | A | G | G | A | A | G | A | A | T | | | |
| 7 | (9) | T | A | G | G | G | T | G | A | A | T | Leu-12Pro | | ND |
| | | | | | | | | | | | | (CTG → CCG: 366) | | |
| | | | | | | | | | | | | Lys168Arg | | |
| | | | | | | | | | | | | (AAG → AGG: 1457) | | |
| 7 | (10) | T | A | G | — | A | A | G | A | A | T | ND | | IVS4 C → T 1243 |
| | | | | | | | | | | | | | | 3′ UTR G → |
| | | | | | | | | | | | | | | A 1607 |
| 8 | (2) | T | A | T | G | A | A | G | A | A | T | ND | | ND |
| 8 | (4) | T | A | G | — | A | A | G | A | A | T | ND | | Asn152 (AAC → |
| | | | | | | | | | | | | | | AAT: 1410) |
| 9 | (1) | T | G | G | G | G | A | G | A | A | T | ND | | IVS4 T → A 1169 |
| | | | | | | | | | | | | | | IVS4 A → G 1219 |
| 9 | (3) | T | A | G | G | G | A | G | A | A | T | ND | | IVS2 A → G 670 |
| | | | | | | | | | | | | | | IVS4 T → A 1169 |
| | | | | | | | | | | | | | | IVS4 T → G 1216 |
| | | | | | | | | | | | | | | 3′ UTR T → |
| | | | | | | | | | | | | | | C 1630 |
| 10 | (1) | T | G | G | G | G | A | G | A | A | T | Lys41Arg | Unpublished | IVS4 T → A 1169 |
| | | | | | | | | | | | | (AAG → AGG: 731) | | |
| 10 | (6) | T | A | T | G | A | A | G | A | A | T | ND | | IVS4 T → A 1169 |
| 11 | (3) | T | A | G | G | G | A | G | A | A | T | ND | | IVS2 A → G 580 |
| | | | | | | | | | | | | | | IVS4 C → T 1101 |
| | | | | | | | | | | | | | | IVS4 T → A 1169 |
| | | | | | | | | | | | | | | IVS1 T → C 303 |
| 11 | (5) | T | A | G | G | A | A | G | A | A | T | ND | | ND |
| 12 | (2) | T | A | G | G | G | A | G | A | A | T | ND | | IVS1 T → C 230 |
| | | | | | | | | | | | | | | IVS2 DelA 508 |
| | | | | | | | | | | | | | | IVS3 T → C 836 |
| | | | | | | | | | | | | | | IVS4 C → T 1101 |
| | | | | | | | | | | | | | | IVS4 T → A 1169 |
| 12 | (4) | T | A | G | G | G | A | G | A | A | T | G → A −48 | Unpublished | IVS4 C → T 1101 |
| | | | | | | | | | | | | | | IVS4 T → A 1169 |
| 13 | (1) | T | A | G | G | G | A | G | A | A | T | ND | | |
| | | | | | | | | | | | | | | (GAA → |
| | | | | | | | | | | | | | | GAG: 1341) |
| 13 | (2) | T | A | G | — | A | A | G | A | A | T | ND | | ND |
| 14 | (1) | T | A | G | G | G | A | G | A | A | T | ND | | IVS4 C → T 1101 |
| | | | | | | | | | | | | | | IVS4 T → A 1169 |
| 14 | (7) | T | A | G | G | A | A | G | A | A | T | ND | | IVS4 T → A 1169 |
| 15 | (1) | T | A | T | G | A | A | G | A | A | T | ND | | ND |
| 15 | (3) | T | A | G | G | G | A | G | A | A | T | ND | | IVS4 T → A 1169 |
| 16 | (2) | T | A | T | G | A | A | G | A | A | T | ND | | ND |
| 16 | (7) | T | A | G | G | A | A | G | A | A | T | Lys41Arg | Unpublished | ND |
| | | | | | | | | | | | | (AAG → AGG: 731) | | |
| 17 | | | | | | | | | | | | | | |
| 18 | (1) | T | A | G | G | A | A | G | A | A | G | C → T −108 | | IVS1 C → T 188 |
| 18 | (2) | T | A | G | G | A | A | G | A | A | T | ND | | IVS1 A → G 124 |
| | | | | | | | | | | | | | | IVS1 A → T 128 |
| 19 | (2) | T | G | G | G | G | A | G | A | A | T | G → A −156 | Unpublished | IVS4 T → A 1169 |
| 19 | (3) | T | A | T | G | A | A | G | A | A | T | ND | | IVS4 T → A 1169 |
| 20 | (2) | T | A | T | G | A | A | G | A | A | T | ND | | IVS4 T → A 1169 |
| | | | | | | | | | | | | | | IVS4 T → C 1208 |
| | | | | | | | | | | | | | | IVS4 T → G 1302 |
| 20 | (3) | T | A | G | G | A | A | G | A | A | T | Asp11Asn | Unpublished | IVS4 T → A 1169 |
| | | | | | | | | | | | | (GAC → AAC: 431) | | |
| 21 | | | | | | | | | | | | | | |
| 22 | (1) | T | A | G | G | G | A | G | A | A | T | ND | | IVS4 T → A 1169 |
| 22 | (2) | T | A | G | G | G | A | G | A | A | T | ND | | IVS4 C → T 1101 |
| | | | | | | | | | | | | | | IVS4 T → A 1169 |
| 23 | (2) | T | A | G | G | A | A | G | A | A | T | T → C −237 | | IVS2 A → G 558 |
| | | | | | | | | | | | | Phe1Leu | | IVS4 T → A 1169 |
| | | | | | | | | | | | | (TTC → CTC: 401) | | |
| 23 | (8) | T | A | G | G | G | A | G | A | A | T | ND | | IVS2 G → A 620 |
| | | | | | | | | | | | | | | IVS4 T → A 1169 |
| 24 | (3) | T | A | T | G | A | A | G | A | A | T | ND | | IVS1 C → A 313 |
| | | | | | | | | | | | | | | IVS4 T → C 1232 |
| 24 | (4) | T | G | G | G | G | A | G | A | A | T | Thr-20Ala | | IVS4 T → A 1169 |
| | | | | | | | | | | | | (ACG → GCG: 341) | | |
| 25 | (1) | T | G | G | G | G | A | G | G | C | T | ND | | IVS3 C → G 879 |
| | | | | | | | | | | | | | | IVS4 T → A 1169 |
| 25 | (4) | T | A | T | G | A | A | G | A | A | T | ND | | IVS2 G → A 685 |
| 26 | (1) | C | A | G | G | G | T | G | A | A | T | ND | | Gly 131 (GGC → |
| | | | | | | | | | | | | | | GGT: 1347) |
| 26 | (2) | T | A | G | G | G | A | G | A | A | T | ND | | IVS1 T → C 281 |
| 27 | (2) | T | A | T | G | A | A | G | A | A | T | Ser43Leu | Unpublished | ND |
| | | | | | | | | | | | | (TCA → TTA: 737) | | |
| 27 | (4) | T | G | G | G | G | A | G | A | A | T | ND | | IVS4 T → A 1169 |
| | | | | | | | | | | | | | | 3′ UTR C → |
| | | | | | | | | | | | | | | T 1659 |
| 28 | (4) | T | A | T | G | G | A | G | A | A | T | ND | | ND |
| 28 | (5) | T | A | G | — | A | A | G | A | A | T | ND | | ND |
| 29 | (3) | T | A | T | G | A | A | G | A | A | T | ND | | ND |
| 29 | (3A) | T | A | G | G | G | A | G | A | A | T | ND | | IVS4 T → A 1169 |
| | | | | | | | | | | | | | | IVS4 A → G 1189 |
| 30 | (2) | T | A | T | G | A | A | G | A | A | T | −177 A → G | | IVS3 T → C 901 |
| | | | | | | | | | | | | Ser108Cys | | IVS4 A → G 1261 |
| | | | | | | | | | | | | (AGC → TGC: 1023) | | |
| | | | | | | | | | | | | Phe176Ser | | |
| | | | | | | | | | | | | (TTC → TCC: 1481) | | |
| 30 | (3) | T | A | T | G | A | A | G | A | A | T | −177 A → G | | IVS1 T → C 234 |
| | | | | | | | | | | | | Ser108Arg | | IVS3 T → C 901 |
| | | | | | | | | | | | | (AGC → CGC: 1023) | | |
| | | | | | | | | | | | | Leu163Pro | | |
| | | | | | | | | | | | | (CTC → CCC: 1442) | | |
| 31 | (3) | T | A | G | G | A | A | G | A | A | T | Lys41Arg | Unpublished | ND |
| | | | | | | | | | | | | (AAG → AGG: 731) | | |
| 31 | (11) | T | A | T | G | A | A | G | A | A | T | Ser108Arg | | IVS3 T → C 901 |
| | | | | | | | | | | | | (AGC → CGC: 1023) | | |
| | | | | | | | | | | | | Phe176Ser | | IVS4 G → A 1097 |
| | | | | | | | | | | | | (TTC → TCC: 1481) | | |
| | | | | | | | | | | | | | | IVS4 A → G 1193 |
| 32-2 | (1) | T | A | G | G | G | A | G | A | A | T | ND | | IVS4 T → A 1169 |
| | | | | | | | | | | | | | | IVS4 A → G 1240 |
| 32-2 | (2) | T | A | T | G | A | A | G | A | A | T | −177 A → G | Unpublished | IVS3 T → C 901 |
| | | | | | | | | | | | | Ser108Arg | Unpublished | |
| | | | | | | | | | | | | (AGC → CGC: 1023) | | |
| | | | | | | | | | | | | Phe176Ser | Unpublished | |
| | | | | | | | | | | | | (TTC → TCC: 1481) | | |
| 32-3 | (3) | T | A | G | G | G | A | G | A | A | T | ND | | Ins GAAA 251 |
| | | | | | | | | | | | | | | IVS4 T → A 1169 |
| 33 | (3) | T | A | T | G | A | A | G | A | A | T | ND | | ND |
| 33 | (4) | T | ? | ? | — | G | A | G | A | A | T | Gene conversion | Unpublished | ND |
| | | | | | | | | | | | | (to GH2), max −161 | | |
| | | | | | | | | | | | | to +69, | | |
| | | | | | | | | | | | | min −86 to −46 | | |
| 34 | (3) | C | A | G | G | A | A | G | A | A | T | ND | | IVS4 T → A 1169 |
| 34 | (4) | T | A | G | G | G | A | G | A | A | T | ND | | IVS4 T → A 1169 |
| 35 | (6) | T | A | G | G | G | A | G | A | A | T | ND | | IVS4 T → A 1169 |
| 35 | (8) | T | G | G | G | G | A | G | A | A | T | ND | | IVS4 T → A 1169 |
| 36 | (3) | T | A | G | G | G | A | G | A | A | T | ND | | IVS1 T → C 281 |
| 36 | (5) | T | A | ? | G | A | A | G | A | A | T | Del5G −57 to −61 | Unpublished | ND |
| | | | | | | | | | | | | Asp26Val | Unpublished | |
| | | | | | | | | | | | | (GAC → GTC: 477) | | |
| 37 | (1) | T | G | G | G | G | A | G | G | C | T | Acceptor splice | Unpublished | IVS4 T → A 1169 |
| | | | | | | | | | | | | site IVS2 | | |
| | | | | | | | | | | | | G → A at −1 (702) | | |
| 37 | (4) | T | A | G | — | G | A | G | A | A | T | Acceptor splice | Unpublished | IVS4 C → T 1212 |
| | | | | | | | | | | | | site IVS2 | | |
| | | | | | | | | | | | | G → A at −1 (702) | | IVS4 T → A 1169 |
| 38 | (2) | T | A | G | — | G | A | G | A | A | T | Acceptor splice | Unpublished | IVS4 T → A 1169 |
| | | | | | | | | | | | | site IVS2 | | |
| | | | | | | | | | | | | G → A at −1 (702) | | |
| 39 | (1a) | T | G | G | G | G | T | G | A | A | T | ND | | IVS4 T→ A 1169 |
| 39 | (3) | T | A | T | G | A | A | G | A | A | T | Thr175Ala | Unpublished* | ND |
| | | | | | | | | | | | | (ACA → GCA: | | |
| | | | | | | | | | | | | 1477)* | | |
| 40 | (1) | T | A | T | G | G | A | G | A | A | T | ND | | ND |
| 40 | (5) | T | A | G | — | A | A | G | A | A | T | ND | | ND |
| 41 | (1) | T | G | G | G | G | A | G | G | C | T | ND | | IVS4 T → A 1169 |
| 41 | (4) | T | A | G | G | G | A | G | A | A | T | | | |
| 41 | (6) | T | A | ? | G | G | A | G | A | A | T | DelG −57 to −61 | Unpublished | |
| 42 | (1) | T | A | G | G | G | A | G | A | A | T | ND | | IVS4 T → A 1169 |
| 42 | (2) | T | A | T | G | A | A | G | A | A | T | ND | | ND |
| 43 | (4) | T | A | G | G | A | A | G | A | A | T | ND | | ND |
| 43 | (6) | C | A | G | G | G | T | G | A | A | T | ND | | IVS4 T → A 1169 |
| 44 | (1) | T | G | G | G | G | A | G | A | A | T | C → T −18 | Unpublished | IVS4 T → A 1169 |
| | | | | | | | | | | | | | | 3′ UTR C → |
| | | | | | | | | | | | | | | T 1536 |
| 44 | (3) | T | A | T | G | A | A | G | A | A | T | Ser85Pro | Unpublished | IVS4 T → A 1169 |
| | | | | | | | | | | | | (TCG → CCG: 954) | | |
| 45 | (4) | T | G | G | G | G | A | G | A | A | T | ND | | IVS1 T → C 281 |
| | | | | | | | | | | | | | | IVS4 T→ A 1169 |
| 46 | (1) | T | A | G | G | G | A | G | A | A | T | ND | | IVS4 T → A 1169 |
| | | | | | | | | | | | | | | IVS4 T → G 1216 |
| 46 | (7) | T | G | G | G | G | A | G | A | A | T | ND | | IVS1 T → C 281 |
| | | | | | | | | | | | | | | IVS4 T → A 1169 |
| 47 | (4) | T | A | G | G | A | A | G | A | A | T | ND | | IVS4 C → T 1101 |
| | | | | | | | | | | | | | | IVS4 T → A 1169 |
| | | | | | | | | | | | | | | 3′UTR T → C 1648 |
| 47 | (7) | T | A | T | G | A | A | G | A | A | T | C → T −347 | | IVS2 A → G 585 |
| | | | | | | | | | | | | A → G −44 | | IVS4 T → A 1169 |
| 48 | (2) | T | A | G | G | G | A | G | A | A | T | ND | | IVS4 T → A 1169 |
| 48 | (5) | T | G | G | G | G | A | G | A | A | G | Thr-24Ala | Miyata et al | IVS4 T → A 1169 |
| | | | | | | | | | | | | (ACA → GCA: 69) | (1997)^{c} | |
| | | | | | | | | | | | | Ala155Val | Unpublished | |
| | | | | | | | | | | | | (GCA → GTA: 1418) | | |
| 49 | (3) | T | A | G | G | A | A | G | A | A | T | ND | | IVS2 T → C 665 |
| 50 | (1) | T | A | G | G | G | A | G | A | A | T | Leu-11Pro | | IVS4 T → A 1169 |
| | | | | | | | | | | | | (CTC → CCC: 369) | | |
| 50 | (2) | T | A | G | G | G | A | G | A | A | T | T → C +31 | | IVS1 A → G 249 |
| | | | | | | | | | | | | Leu-11Pro | | IVS4 T → A 1169 |
| | | | | | | | | | | | | (CTC → CCC: 369) | | |
| | | | | | | | | | | | | Ile4Val | | |
| | | | | | | | | | | | | (ATT → GTT: 410) | | |
| 50 | (3) | T | A | G | G | G | A | G | A | A | T | Met-26Val | | IVS4 T → A 1169 |
| | | | | | | | | | | | | (ATG → GTG: 63) | | |
| | | | | | | | | | | | | Leu-11Pro | | |
| | | | | | | | | | | | | (CTC → CCC: 369) | | |
| 51 | (2) | T | A | G | G | G | A | G | A | A | T | Leu-11Pro | | IVS1 C → G 226 |
| | | | | | | | | | | | | (CTC → CCC: 369) | | |
| | | | | | | | | | | | | | | IVS4 T → A 1169 |
| 51 | (3) | T | A | T | G | A | A | G | A | A | T | ND | | ND |
| 52 | (1) | T | A | G | G | G | A | G | A | A | T | Lys168Glu | | IVS4 T → A 1169 |
| | | | | | | | | | | | | (AAG → GAG: 1456) | | 3′ UTRT → C 1558 |
| 52 | (2) | T | A | G | G | G | A | G | A | A | T | C → T −56 | | IVS4 T → A 1169 |
| | | | | | | | | | | | | Donor splice | | 3′ UTR T → |
| | | | | | | | | | | | | site IVS23 | | C 1558 |
| | | | | | | | | | | | | T → Cat +2 (824) | | |
| | | | | | | | | | | | | Lys168Glu | | |
| | | | | | | | | | | | | (AAG → GAG: 1456) | | |
| 53 | (1) | T | A | G | G | A | A | G | A | A | T | Ser71Phe | Unpublished | ND |
| | | | | | | | | | | | | (TCC → TTC: 821) | | |
| 53 | (3) | T | A | T | G | A | A | G | A | A | T | ND | | IVS2 G → A 676 |
| | | | | | | | | | | | | | | IVS4 C → T 1182 |
| 54 | (1b) | T | A | G | G | G | A | G | A | A | T | ND | | IVS1 T → C 284 |
| | | | | | | | | | | | | | | IVS2 A → G 573 |
| | | | | | | | | | | | | | | IVS4 T → A 1169 |
| 54 | (2) | T | A | G | G | G | A | G | A | A | T | ND | | IVS1 A → G 134 |
| 55 | (2) | T | ? | ? | — | G | A | G | A | A | T | Gene conversion | Unpublished | ND |
| | | | | | | | | | | | | (to GH2), max −161 | | |
| | | | | | | | | | | | | to +69, | | |
| | | | | | | | | | | | | min −86 to −46 | | |
| 55 | (3) | T | A | G | — | G | A | G | A | A | T | Acceptor splice | Unpublished | IVS4 T → A 1169 |
| | | | | | | | | | | | | site IVS2 | | |
| | | | | | | | | | | | | G → A at −1 (702) | | |
| 56 | (1) | T | A | G | — | G | A | G | A | A | T | Acceptor splice | Unpublished | IVS4 T → A 1169 |
| | | | | | | | | | | | | site IVS2 | | |
| | | | | | | | | | | | | G → A at −1 (702) | | |
| 56 | (1A) | T | A | T | G | A | A | G | A | A | T | A → G −24 | | ND |
| 57 | (1) | T | A | G | — | G | A | G | A | A | T | Acceptor splice | Unpublished | IVS4 T → A 1169 |
| | | | | | | | | | | | | site IVS2 | | |
| | | | | | | | | | | | | G → A at −1 (702) | | |
| 57 | (2) | T | A | G | G | G | A | G | A | A | T | Asp107Gly | Unpublished | IVS4 T → A 1169 |
| | | | | | | | | | | | | (GAC → GGC: 1021) | | |
| 58 | (1) | T | A | G | G | G | A | G | A | A | T | ND | | IVS4 T → A 1169 |
| 58 | (4) | T | ? | ? | — | G | A | G | A | A | T | Gene conversion | Unpublished | ND |
| | | | | | | | | | | | | (to GH2), max −161 | | |
| | | | | | | | | | | | | to +69, | | |
| | | | | | | | | | | | | min −86 to −46 | | |
| 59 | (1) | T | A | G | G | G | A | G | G | A | T | ND | | IVS4 C → T 1101 |
| | | | | | | | | | | | | | | IVS4 T → A 1169 |
| 59 | (5) | T | A | G | G | G | A | G | A | A | T | ND | | IVS4 T → A 1169 |
| 60 | (2) | T | A | G | — | G | A | G | A | A | T | Acceptor splice | Unpublished | IVS4 T → A 1169 |
| | | | | | | | | | | | | site IVS2 | | |
| | | | | | | | | | | | | G → A at −1 (702) | | |
| 60 | (4) | T | A | G | G | G | A | G | A | A | T | G → A −280 | | IVS1 T → C281 |
| | | | | | | | | | | | | | | IVS4 T → A 1169 |
| 61 | (1) | T | A | T | G | A | A | G | A | A | T | ND | | ND |
| 61 | (4) | T | A | G | — | G | A | G | A | A | T | Acceptor splice | | ND |
| | | | | | | | | | | | | site IVS2 | | |
| | | | | | | | | | | | | G → A at −1 (702) | | |
| 62 | (1) | T | A | G | G | G | A | G | A | A | T | ND | | IVS4 T → A 1169 |
| 62 | (2) | C | A | G | G | G | A | G | A | A | T | Gln91Leu | | IVS4 T → A 1169 |
| | | | | | | | | | | | | (CAG → CTG: 973) | | |
| 63 | (2) | T | A | G | G | G | A | G | A | A | T | Glu74Lys | | IVS4 T → A 1169 |
| | | | | | | | | | | | | (GAG → AAG: 921) | | |
| 63 | (3) | T | A | T | G | A | A | G | A | A | T | ND | | ND |
| 64 | (7) | T | G | G | G | G | A | G | A | A | T | Glu56Gly | | IVS4 T → A 1169 |
| | | | | | | | | | | | | (GAG → GGG: 776) | | |
| 64 | (8) | T | A | T | G | A | A | G | A | A | T | ND | | IVS3 C → A 883 |
| 65 | (1) | T | A | T | G | A | A | G | A | A | T | A → G −248 | Unpublished | IVS4 T → A 1169 |
| 65 | (2) | T | A | T | G | A | A | G | A | A | T | ND | | IVS2 G → T 586 |
| | | | | | | | | | | | | | | IVS3 T → C 836 |
| | | | | | | | | | | | | | | IVS4 T → A 1169 |
| 66 | (1) | T | A | G | G | G | A | G | A | A | T | Leu-11Pro | | IVS1 T → A 284 |
| | | | | | | | | | | | | (CTC → CCC: 369) | | |
| | | | | | | | | | | | | | | IVS4 T → A 1169 |
| 66 | (2) | T | A | G | G | G | A | G | A | A | T | Val110Ile | | IVS4 T → A 1169 |
| | | | | | | | | | | | | (GTC → ATC: 1029) | | |
| 67 | (2b) | T | G | G | G | G | A | G | G | C | T | G → A −364 | | IVS1 T → C 303 |
| | | | | | | | | | | | | | | IVS4 T → A 1169 |
| 67 | (13) | T | A | G | G | G | A | G | A | C | T | T → C −413 | | IVS1 G → T 135 |
| | | | | | | | | | | | | | | IVS1 G → C 236 |
| | | | | | | | | | | | | | | IVS4 T → A 1169 |
| 67 | (15) | T | A | G | G | G | A | G | A | A | T | ND | | IVS2 T → C 649 |
| 68 | (2b) | T | A | T | G | A | A | G | A | A | T | Tyr143His | | ND |
| | | | | | | | | | | | | (TAC → CAC: 1381) | | |
| 68 | (7) | T | A | T | G | A | A | G | A | A | T | ND | | IVS2 A → T 519 |
| | | | | | | | | | | | | | | IVS2 G → A 524 |
| | | | | | | | | | | | | | | 3′UTR T → C 1654 |
| 69 | (3) | T | A | T | G | A | A | G | A | A | T | T → C −495 | | IVS1 A → G 136 |
| | | | | | | | | | | | | | | IVS3 T → C 839 |
| 69 | (11) | T | A | T | G | A | A | G | A | A | T | Glu30Gly | | Tyr103 |
| | | | | | | | | | | | | (GAG → GGG: 489) | | (TAC → |
| | | | | | | | | | | | | | | TAT: 1010) |
| | | | | | | | | | | | | | | IVS4 T → G 1196 |
| 70 | (5) | T | A | T | G | A | A | G | A | A | T | T → C −30 | | ND |
| 70 | (10) | T | G | G | G | G | A | G | G | C | T | A → G −267 | | IVS4 T → A 1169 |
| | | | | | | | | | | | | A → G −248 | | 3′UTR T → C 1654 |
| | | | | | | | | | | | | Gln22Arg | | |
| | | | | | | | | | | | | (CAG → CGG: 465) | | |
| | | | | | | | | | | | | Lys41Arg | | |
| | | | | | | | | | | | | (AAG → AGG: 731) | | |
| | | | | | | | | | | | | Trp86Arg | | |
| | | | | | | | | | | | | (TGG → CGG: 957) | | |

Key:
IVS: intervening sequence (intron)
ND: no mutation or polymorphism detected
UTR: untranslated region
^{a}Patient (clone number)
^{b}Nucleotide numbering based on GH1 reference sequence. At −31, alternative alleles are presence or absence of a G.
^{c}Amino acid residue number and substitution, (nucleotide substitution and number based upon GH1 reference sequence).
^{d}IVS number, nucleotide change, base number
^{e}Miyata I, Cogan J, Prince MA, Kamijo T, Ogawa M, Phillips JA Detection of growth hormone defects by dideoxy fingerprinting (ddF) Endocrinol J. 44 149-154 (1997).
*The first time this mutation has been identified in vivo; previously identified in vitro as a result of alanine scanning mutagenesis (Cunningham et al USP 5 849 535 (1998)).

The GHJ reference sequence is derived from Chen et al. (1989) which was accessed through Genbank (Accession Number: J03071). Of 68 patients so far analysed, mutations have been found in 47 of them. All mutations detected were found in the heterozygous state with the exception of patients 30, 37, 50 and 52 (homozygous), patients 30, 31, 44, 50, 52, 55, 56, 57, 60, 66 and 67 (compound heterozygous for non-identical lesions in trans) (ie on different alleles) and patients 7, 23, 30, 31, 32, 36, 47, 48, 50, 52 and 70 who possess 2 or more mutations in cis (ie on the same allele).

(a) Missense Mutations

A total of 31 novel single base-pair substitutions have been noted within the coding region of the GH1 gene that served to change the amino acid encoded. Evidence for the pathological involvement of these missense mutations came from four sources: (i) the study of a control population, (ii) the nature of the amino acid substitutions and the degree of evolutionary conservation of the residues in question, (iii) molecular modelling and (iv) the in vitro assay of their signal transduction activities.

(i) Studies of GH1 Coding Sequence Variation in Controls

A total of 80 healthy British controls of Caucasian origin were screened for variants within the coding region of the GH1 gene. Five examples of silent substitutions found in single patients were noted [GAC→GAT at Asp26, TCG→TCC at Ser85, TCG→TCA at Ser85, ACG→ACA at Thr123 and AAC→AAT at Asn109]. In addition, two missense substitutions were noted [AAC→GAC, Asn47→Asp; GTC→ATC, Va110→Ile, 4/160 alleles]; only the Val110→Ile substitution had been found in our patient study (patient 66). Molecular modelling suggested that this substitution exerts a deleterious effect on the structure of GH; Val110 forms part of the hydrophobic core at the N-terminal end of helix 3 and its replacement by Ile with its longer sidechain would cause steric hindrance. It may thus be that while the Val110→Ile substitution occurs relatively frequently in both control and patient populations, it is nevertheless capable of influencing stature. This notwithstanding, the relative paucity of missense mutations in the control population argues in favour of the authenticity of the lesions found in the patient cohort.

(ii) Nature of the Amino Acid Substitution and Evolutionary Conservation of the Residue Involved

The probability that a missense mutation will come to clinical attention depends upon a number of factors including the sequence structure of the gene in question, the magnitude of the amino acid substitution, the precise location and immediate environment of the substituted residue within the protein molecule, and its resulting effects on the structure and function of the protein (Wacey et al Hum Genet 94 594-608 (1994)). In order to assess whether the missense mutations detected are likely to be significant pathologically, the biophysical properties of the changes were examined individually (Table 7C). In most cases, the changes were non-conservative in that the substituting amino acid differed markedly from the substituted amino acid, thereby supporting the contention that they are of pathological significance.

Evidence for the involvement of missense mutations in pathology can be derived from evolutionary conservation data, since those amino acid residues that are evolutionarily conserved are likely to possess a biological function. Conversely, those residues that are not conserved evolutionarily are less likely to be of functional significance. Pathological lesions tend therefore to occur in evolutionarily conserved residues whereas neutral polymorphisms or rare variants do not (Wacey et al, ibid). Each of the human GH residues found to be involved in missense mutation was therefore examined in terms of its evolutionary conservation through comparison with the orthologous GH protein sequences of 19 other vertebrates (Table 7C). The majority of residues affected by missense mutation were found to be highly, sometimes strictly, conserved, again supporting the view that these lesions are of pathological significance.

[Table 7C follows]

TABLE 7C
Missense mutations, biophysical properties and
evolutionary conservation of residues involved

| | Biophysical | |
|---|---|---|
| | properties of | |
| | change | |
| Amino acid | (conservative/ | Evolutionary conservation of amino |
| substitution | non-conservative) | acid residue in vertebrate GH proteins |
| Met→Val −26 | | Initiator methionine |
| Thr→Ala −24 | NC: polar→hydrophobic | Conserved in mouse, otherwise hydrophobic Ala in |
| | | most mammals |
| Thr→Ala −20 | NC: polar→hydrophobic | Conserved in arterodactyls, rabbit, rodents. |
| | | Otherwise polar in dog (Asn) and birds (Ser). |
| | | Gly in frog. |
| Leu→Pro −12 | C: hydrophobic | Conserved in all mammals and most fish but not |
| | | birds (polar Thr) or frog (Val). |
| Leu→Pro −11 | C: hydrophobic | Conserved in all mammals, birds, rock cod. |
| | | Hydrophobic residue in other fish |
| Phe→Leu 1 | C: hydrophobic | Conserved in mammals, birds, frog but not fish |
| | | (polar Tyr, Gln and Gly) |
| Ile→Val 4 | C: hydrophobic | Not conserved except in salmon. Met in other |
| | | mammals, birds, frog |
| Asp→Asn 11 | NC: charged→polar | Not conserved. Hydrophobic Ala in most other |
| | | mammals and birds. |
| Gln→Arg 22 | NC: polar→charged | Conserved in most other mammals (Glu in whale), |
| | | frog and some fish. Hydrophobic Leu in birds, |
| | | turtle and some fish. |
| Asp→Val 26 | NC: charged→hydrophobic | Conserved in mammals, turtle, frog. Charged in |
| | | birds (Glu) and fish (Arg/Lys) |
| Glu→Gly 30 | NC: charged→small, | Conserved in mammals, birds, turtle and rock cod. |
| | uncharged | Charged Asp in frog and most fish |
| Lys→Arg 41 | C: charged | Arg in all other vertebrates! |
| Ser→Leu 43 | NC: polar→hydrophobic | Conserved in all mammals except whale (Phe), |
| | Increase in side-chain | turtle, frog and shark. Birds (Thr), bony fish (Leu) |
| Glu→Gly 56 | NC: charged→small | Conserved in mammals, birds, turtle, frog, shark. |
| | uncharged | Charged Asp in bony fish |
| Arg→Gly 64 | NC: charged→small | Strictly conserved in all other vertebrates (charged |
| | uncharged | Lys) |
| Ser→Phe 71 | NC: polar→hydrophobic. | Conserved in all vertebrates except rodents (polar |
| | Increase in side-chain | Thr) |
| Glu→Lys 74 | C: charged | Conserved in mammals, birds, turtle, frog, shark. |
| | | Charged Lys in fish. |
| Ser→Pro 85 | NC: polar→hydrophobic | Strictly conserved in all vertebrates |
| Trp→Arg 86 | NC: polar→charged | Strictly conserved in all vertebrates |
| Gln→Leu 91 | NC: polar→hydrophobic | Conserved in all vertebrates except sea bream and |
| | | rock cod (charged Arg) |
| Asp→Gly 107 | NC: charged→small | Conserved in all vertebrates except bony fish |
| | uncharged | (Arg/Ala/Pro) |
| Ser→Cys 108 | C: polar | Arg (charged) in most other vertebrates except fish |
| | | (polar Asn) |
| Ser→Arg 108 | NC: polar→charged. | Arg (charged) in most other vertebrates except fish |
| | Increase in side-chain | (polar Asn) |
| Val→Ile 110 | C: hydrophobic | Conserved in most other vertebrates except bony |
| | | fish (hydrophobic Ile) |
| Tyr→His 143 | C: polar | Conserved in vertebrates except carp and |
| | | goldfish (hydrophobic Phe) |
| Ala→Val 155 | C: hydrophobic | Conserved in all mammals, birds, turtle and |
| | | shark. Gly in frog, Ala or polar Ser/Asn in |
| | | bony fish |
| Leu→Pro 163 | C: hydrophobic | Strictly conserved in all vertebrates |
| Lys→Arg 168 | C: charged | Strictly conserved in all vertebrates |
| Lys→Glu 168 | C: charged | Strictly conserved in all vertebrates |
| Thr→Ala 175 | NC: polar→hydrophobic | Strictly conserved in all vertebrates |
| Phe→Ser 176 | NC: hydrophobic→ | Polar Tyr in all other vertebrates |
| | polar | |

Orthologous GH proteins compared
(% identical, % conservatively changed vs human in brackets)
Mouse (66, 77), rat (64, 75), rabbit (66, 77), whale, dog (67, 78), pig (67, 78), sheep (66, 76), cow (66, 76), turkey (55, 74), chicken (56, 73), duck (55, 72), turtle, frog (45, 68), shark, sea bream, rock cod, salmon, carp (38, 57), goldfish (37, 57).

(iii) Missense Mutations with Putative Functional Consequences as Adduced by Molecular Modelling

Molecular modelling studies suggested that the missense mutations are often located in regions of the GH molecule that either interact with the GH receptor or which may influence GH-GH receptor interactions. Missense mutations were modelled by simple replacement of the appropriate amino acid residue in the X-ray crystallographic structure of human growth hormone. The wild-type and mutant “structures” were then compared with respect to electrostatic interactions, hydrogen bonding, hydrophobic interactions and surface exposure. The majority of missense mutations appeared to result from structural deformation of the GH molecule rather than functional perturbation. Such amino acid substitutions might result in improper folding or instability of the molecule. However, the following 8 missense mutations appeared to be reasonable candidates for amino acid substitutions with functional as opposed to purely structural consequences:

Ile4Val: N-terminal, within site 2. Alanine scanning mutagenesis (ASM) has previously demonstrated that replacement of Ile4 affected GHR dimerization.

Gln22Arg: Helix 1. Introduction of Arg leads to loss of H-bond with Asp26. It also leads to the introduction of two positive charges on same side of helix. May destabilize helix formation or may create unfavourable interaction with Arg217 of GHR.

Lys41Arg: Loop 1. Lys41 solvent accessible. Orthologous genes often possess Arg at analogous location. Lys41 Nζ forms H-bonds with GH residues Tyr28 and Glu32 and exhibits an ionic interaction with GHR Glu127 Oε2. Lys41 implicated in GHR binding by ASM. Introduction of Arg probably does not increase affinity of GH for GHR. Subtle change, not necessarily pathological. Normal GH levels in patients.

Glu56Gly: Glu56 in loop region between helices 1 and 2, and comprises part of binding site 1. Glu56 interacts with Arg71 of GHR. Glu56 also interacts internally with Lysl68 which forms part of the binding energy hotspot in GH-GHR complexes.

Arg64Gly: Loop 2. Arg64 solvent accessible. Arg or Lys conserved at this location. Arg64 implicated in GHR binding by ASM. Basic Arg sidechain forms salt-bridge with, and H-bonds to, GHR Aspl64. Arg64 also exhibits hydrophobic interaction with Trpl69 of GHR. Replacement by Gly will weaken GHR binding and may destabilize helix. Normal GH level in patient.

Lys168Arg: Helix 4. Hydrophobic interaction between Lys168 and Trp104 of GHR. No adverse interactions predicted. High normal GH in patient.

Lys168Glu: Lys168 exhibits extensive hydrophobic interactions with Trp104 of GHR. Charge may stabilize active conformation of GH by forming favourable intramolecular electrostatic interactions. Substitution with Glu may not have severe effect on activity.

Thr175Ala: Helix 4. Thr175 implicated in GHR binding by ASM; Thr175 forms H-bond with Asp171 of GH and Trp169 and Arg 43 of GHR. Introduction of Ala may destabilize helix thereby decreasing receptor binding.

The above-noted missense mutations might provide an indication of the presence of a naturally-occurring growth hormone inhibitor, which—but for the selection criteria applied according to the present invention—might never have come to light.

(iv) Assay of Signal Transduction Activity of GH Variants

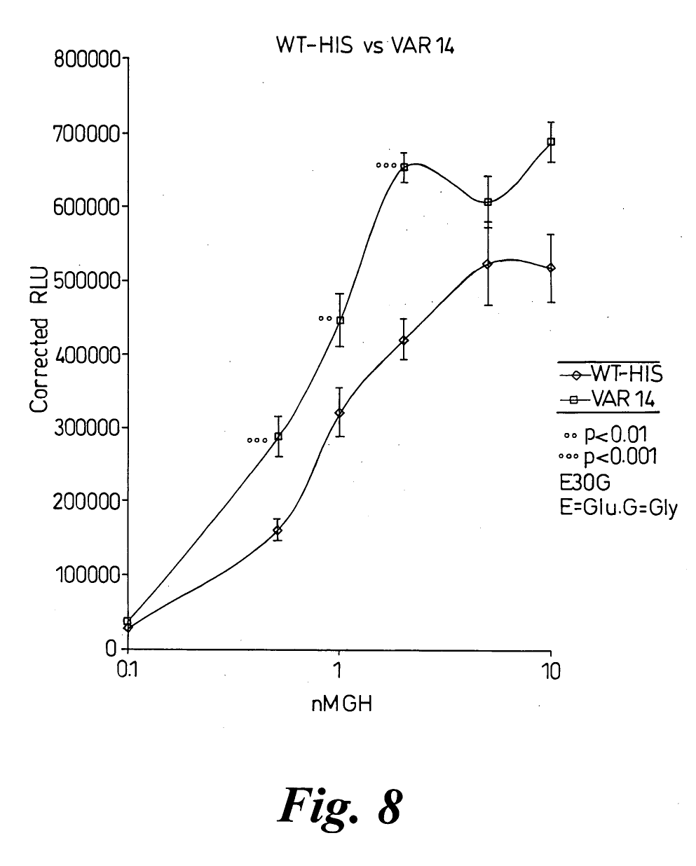

A luciferase reporter gene assay system (according to the method of Ross R J M et al in Molec Endocrin 11 265-73 (1997)) was used to assay the signal transducing activity (biological activity) of the GH variants. For growth hormone to be biologically active, it must bind to two GH receptors and cause receptor dimerization. This then causes the activation of an intracellular tyrosine kinase known as JAK2. JAK2, in turn, phosphorylates and thus activates the transcription factor STAT 5. Phosphorylated STAT 5 dimerizes, translocates to the nucleus and binds to STAT 5-responsive promoters thereby switching on the expression of GH-responsive genes. The assay of GH biological activity that we have used requires all stages of this pathway to be functional. It can be seen from Table 7D that some variants eg Q22R, K41R, W86R and S108R are associated with a dramatically reduced ability to activate the JAK/STAT signal transduction pathway. The variant E30G, patient 69, has significantly enhance ability to activate the JAK/STAT signal transduction pathway, thereby acting as a super-agonist (data shown in FIG. 8, where RLU signifies Relative Light Units).

TABLE 7D
Assay of signal transduction activity of GH variants

| Patient No. | Mutation | % WT | SEM | p vs WT |
|---|---|---|---|---|
| Wild-type | — | 100 | 3 | |
| 48 | T-24A | 92 | 5 | NS |
| 23 | F1L | 121 | 4 | NS |
| 70 | Q22R | 49 | 2 | 0.001 |
| 36 | D26V | 85 | 5 | NS |
| 69 | E30G | 137 | 6 | 0.001 |
| 10, 16, 31, 70 | K41R | 67 | 5 | 0.001 |
| 27 | S43L | 93 | 6 | NS |
| 53 | S71F | 77 | 9 | 0.05 |
| 44 | S85P | 78 | 7 | 0.05 |
| 70 | W86R | 75 | 3 | 0.001 |
| 57 | D107G | 100 | 3 | NS |
| 30, 31, 32 | S108R | 46 | 5 | 0.001 |
| 48 | A155V | 85 | 9 | NS |
| 3 | L163P | 92 | 6 | NS |
| 7 | K168R | 100 | 8 | NS |
| 39 | T175A | 67 | 6 | 0.01 |
| 30, 31, 32 | S108R + F176S | 34 | 2 | 0.001 |
| 70 | Q22R + K41R + W86R | 53 | 2 | 0.001 |

Results are expressed as % activity as compared to wild-type at a dose of 1 nM in the luciferase reporter gene assay (1 nM=approx ED50 of wild-type GH in the assay). p indicates the probability that the difference between what is observed and what occurs in the wild type is significant. NS indicates ‘not significant’.

One missense mutation (Lys41Arg) has been found in four unrelated patients, three of whom have different haplotype backgrounds. This is consistent with recurrent mutation (ie. independent mutation events) at this site. The IVS2 G→A transition at position −1 mutation was found in a total of eight alleles in 8 apparently unrelated patients; since two distinct haplotypes are evident, at least two examples of this lesion are likely to have been recurrent whilst the remainder may be identical-by-descent. Three examples of the promoter gene conversion event were also noted in this patient sample. Multiple examples of various other lesions were also noted [A→G −177 (3), A→G −248 (2), Leu-Pro (4), Ser108Arg (2), Lys168Glu (2), Phe176Ser (2) and Leu163Pro (2)]. In total, 10 recurring mutations correspond to 32/75 (43%) mutant alleles found in our patient sample. This is very encouraging in terms of the prospect for the rapid detection of frequent pathological lesions in the GH1 gene.

(b) Promoter Haplotypes

In our study, 15/17 of the known polymorphic nucleotides within the GH1 gene promoter were found to vary. Variation at these 15 positions was ascribed to a total of 40 different haplotypes in our patient and control (157 British army recruits of Caucasian origin) populations. These haplotypes varied in frequency (Table 7F) from 0.339 (haplotype 1), to 0.0033 (haplotypes 25-36), to 0 (haplotypes 37-40, which were patient-specific in that they were found in the patient but not the control population).

We have found that these promoter haplotypes differ with respect to their ability to drive luciferase gene expression in a reporter gene assay. 27 of the 40 haplotypes have so far been studied in rat pituitary GH3 cells. For each haplotype, 6 replicates were performed in 3 different experiments (ie 18 replicates in total). Those haplotypes that are associated with a significantly reduced level [<62% that of the most common haplotype (no. 1)] of luciferase reporter gene expression (and which could therefore be associated with a reduced level of GH1 gene expression in vivo) are listed in Table 7E, together with their respective frequencies in our patient and control populations.

These findings suggest that ˜15% of individuals in the normal population may be heterozygous for a GH1 promoter haplotype that is (at least in vitro) associated with a level of GH synthesis >40% lower than that associated with the possession of the most common haplotype. Further, it may be that some 2% of the normal population may possess two such low expressing haplotypes (either identical or non-identical) and could, as a direct result, exhibit significantly lower than average GH levels. If in vivo studies support this contention, then it may be that a diagnostic screening strategy should incorporate promoter haplotype determination as well as mutation detection.

TABLE 7E
Promoter haplotypes; their frequency and relative strength as
measured in a luciferase reporter gene assay

| | |
|---|---|
| | Frequency of |
| Luciferase | haplotypes (%) |

| Haplotype | activity ± sem | controls | patients |
|---|---|---|---|
| 1 | 100 ± 18 | 33.9 | 26.4 |
| 3 | 59 ± 15 | 9.2 | 8.5 |
| 5 | 57 ± 13 | 4.3 | 5.4 |
| 10 | 61 ± 18 | 2.0 | 0.0 |
| 23 | 28 ± 15 | 1.0 | 0.8 |
| 26 | 55 ± 26 | 0.3 | 0.8 |
| 29 | 62 ± 15 | 0.3 | 0.0 |

TABLE 7F
Summary of the different promoter haplotypes found in the GH1 genes of controls during the course of our study

| | −476 | −339 | −308 | −301 | −278 | −168 | | −57 | −31 | | −1 | +3 | | +26 | +59 | Frequency |
|---|---|---|---|---|---|---|---|---|---|---|---|---|---|---|---|---|
| Haplotype | (G/A) | (ΔG) | (T/G) | (T/G) | (G/T) | (T/C) | −75 (A/G) | (G/T) | (ΔG) | −6 (G/A) | (T/A/C) | (G/C) | +16 (A/G) | (A/C) | (T/G) | (%) |
| 1 | G | G | G | G | G | T | A | T | G | A | A | G | A | A | T | 33.9 |
| 2 | G | G | G | G | T | T | A | G | G | G | A | G | A | A | T | 16.5 |
| 3 | G | G | T | T | G | T | A | G | G | A | A | G | A | A | T | 9.2 |
| 4 | G | G | T | T | G | T | A | G | — | A | A | G | A | A | T | 5.3 |
| 5 | G | G | G | G | T | T | G | G | G | G | A | G | A | A | T | 4.3 |
| 6 | G | G | T | T | G | T | A | G | — | A | A | G | A | A | G | 3.0 |
| 7 | G | G | G | G | T | T | A | G | G | G | T | G | A | A | T | 2.6 |
| 8 | G | G | T | T | G | T | A | G | G | G | A | G | A | A | T | 2.0 |
| 9 | G | G | G | G | T | T | A | T | G | G | A | G | A | A | T | 2.0 |
| 10 | G | G | T | T | G | T | A | G | — | G | A | G | A | A | T | 2.0 |
| 11 | G | G | G | G | T | T | G | G | G | G | A | G | G | C | T | 1.6 |
| 12 | G | G | G | G | T | T | A | G | G | A | A | G | A | A | T | 1.6 |
| 13 | G | — | G | G | T | T | G | G | G | G | A | G | A | A | T | 1.6 |
| 14 | G | G | G | G | T | C | A | G | G | G | T | G | A | A | T | 1.6 |
| 15 | G | G | T | T | G | T | A | G | G | G | T | G | A | A | T | 1.3 |
| 16 | G | G | G | G | T | T | G | G | G | A | A | G | A | A | T | 1.3 |
| 17 | G | — | G | G | T | T | A | G | G | G | A | G | A | A | T | 1.3 |
| 18 | G | G | G | G | T | T | A | G | — | G | A | G | A | A | T | 0.99 |
| 19 | A | G | G | G | T | T | A | G | G | G | A | G | A | A | T | 0.99 |
| 20 | G | G | G | G | G | T | A | G | — | A | A | G | A | A | T | 0.99 |
| 21 | G | G | G | G | T | T | G | G | G | G | A | G | A | A | G | 0.99 |
| 22 | G | G | T | T | G | T | A | T | G | A | A | G | A | A | T | 0.99 |
| 23 | G | G | G | G | G | T | A | G | G | A | A | G | A | A | T | 0.99 |
| 24 | G | G | T | T | G | T | G | G | — | A | A | G | A | A | T | 0.66 |
| 25 | G | G | T | T | G | T | A | G | G | A | A | G | A | A | G | 0.33 |
| 26 | G | G | G | G | T | T | G | G | G | G | T | G | A | A | T | 0.33 |
| 27 | G | G | G | G | T | T | A | T | G | A | A | G | A | A | T | 0.33 |
| 28 | G | G | G | G | T | T | A | G | — | A | A | G | A | A | T | 0.33 |
| 29 | A | G | G | G | T | T | A | G | G | A | A | G | A | A | T | 0.33 |
| 30 | G | — | G | G | T | T | A | G | G | A | A | G | A | A | T | 0.33 |
| 31 | G | G | G | G | T | T | G | G | — | G | A | G | A | A | T | 0.33 |
| 32 | G | G | T | T | G | T | G | G | G | G | A | G | A | A | G | 0.33 |
| 33 | G | G | G | G | T | T | A | G | G | G | A | G | G | C | T | 0.33 |
| 34 | G | — | G | G | T | C | A | G | G | G | T | G | A | A | T | 0.33 |
| 35 | G | G | G | G | G | T | A | G | G | A | C | C | A | A | T | 0.33 |
| 36 | G | G | G | G | T | T | A | G | G | G | T | G | A | A | G | 0.33 |
| 37 | A | G | G | G | T | T | A | G | G | G | A | G | G | A | T | 0 |
| 38 | G | G | G | G | T | C | A | G | G | A | A | G | A | A | T | 0 |
| 39 | G | G | T | T | G | T | A | G | G | G | A | G | A | C | T | 0 |
| 40 | G | G | G | G | T | C | A | G | G | G | A | G | A | A | T | 0 |

Frequencies given are derived from the control (157 British army recruits of Caucasian origin) population.

(c) Promoter Mutations

Various novel promoter variants (eighteen single base-pair substitutions, two micro-deletions and an extensive gene conversion event) were detected in our patient cohort. Evidence for the authenticity of these lesions was sought by (i) studying the GHI promoter region in healthy controls, (ii) studying the degree of evolutionary conservation of the nucleotides affected in different mammalian species and (iii) determining their effect on GH1 promoter function in vitro by means of a luciferase reporter gene assay.

(i) GH1 Promoter Variants in Controls

The GH1 promoter region was screened for mutations in 157 healthy British controls of Caucasian origin. The only sequence change noted which corresponded to a mutation found in the patient sample was a G→A transition at 48 which was detected in 2 individuals. Three further substitutions specific to the control sample were found in single individuals (+62 A→G, −123 T→C and −373 G→A). Finally, a gene conversion event (minimum −57 to −31, maximum −168 to −6) was noted in a single individual which was also specific to the control sample. Thus, many fewer changes were detected in the controls than in the patients, a finding consistent with the patient mutations being of pathological significance.

(ii) Evolutionary Conservation

DNA sequence, corresponding to 130 bp upstream of the transcriptional initiation site of the GH1 gene, was available from 10 mammalian species. Where ascertainment was possible, the nucleotides found to be mutated in patients were evolutionarily conserved in 7/10 cases (+31 TIC, −18 COT, −24 AUG, −30 T→C, Δ5G −57 to −61, ΔG −57 to −61, and −108 C→T). This finding is consistent with the functional importance of the nucleotides found to be mutated in our patient cohort.

(iii) Luciferase Reporter Gene Analysis of GH1 Promoter Mutations

The various putative promoter mutations were compared in terms of their ability to drive luciferase gene expression in a reporter gene assay (Table 7G). For each haplotype, 6 replicates were performed in 3 different experiments (ie 18 replicates in total) in both rat pituitary GH3 cells and human HeLa cells. Significantly lower than normal expression levels were noted for the T→C −30 transition and the Δ5G −57 to deletion in HeLa cells (a tendency also noted in GH3 cells). Thus, reporter gene expression assays were supportive of the pathological involvement of these two lesions.

TABLE 7G
Putative Promoter Mutations v Reporter Gene Expression

| | | Luciferase | |
|---|---|---|---|
| | | activity | Normalized |
| Promoter | Associated | Normalized | haplotype ± sem |
| mutation | haplotype | to GH3 | HeLa |
| A→G −248 | 1 | 115 ± 16 | 105 ± 18 |
| T→C −495 | 1 | 127 ± 11 | 106 ± 15 |
| A→G −177 | 1 | 98 ± 13 | 166 ± 10 |
| T→C −30 (TATA) | 1 | 86 ± 16 | 57 ± 19 |
| A→G −24 | 1 | 117 ± 19 | 113 ± 13 |
| C→T −347, A→G −44 | 1 | 166 ± 20 | 144 ± 12 |
| A→G +62 | 1 | 130 ± 10 | 112 ± 15 |
| G→A −48, A→G −498 | 2 | 90 ± 16 | 107 ± 18 |
| T→C −508 | 2 | 117 ± 17 | 99 ± 11 |
| ΔGGGGG −57 to −61 | 2 | 91 ± 16 | 48 ± 14 |
| ΔG −57 | 2 | 106 ± 19 | 96 ± 16 |

(d) Mutations Affecting mRNA Splicing

Two novel variants in splice sites were noted, one T→C transition in the donor splice site of exon 3, the other a common single base-pair substitution in the obligate AG dinucleotide of the exon 2 acceptor splice site. The latter mutation has been further characterized by means of an in vitro splicing assay; evidence for its pathogenicity comes from the observation that, under assay conditions, it leads to the “skipping” (exclusion) of exon 3 from the GH1 MRNA transcript.

(e) Polymorphisms in the Human GH1 Gene

During the course of our study, some 71 different putative polymorphisms were identified within the exons, introns or 3′ untranslated region (3′UTR) of the GH1 gene (Table 7A). Most occurred only once and may be rare variants. All except the IVS4 T→A 1169 polymorphism, reported by Hasegawa et al (ibid), are novel. IVS1-4 denote intron locations:

(f) Locus Control Region Polymorphisms

A total of 11 putative polymorphisms were found in the locus control region. These were 154 G→A, 154 G→C, 457 G→A, 505 G→T, 507 T→G, 661 C→T, 1055 C→T, 1429 C→G, 1568 T→G, 1615-1620 ΔGGTGGT and 1934 T→C. Numbering follows the reference sequence in FIG. 4. Taken together, no significant difference in allele frequency was noted between the patient and control groups. However, the 505 G→T, 1055 C→T and 1934 T→C substitutions were patient-specific and could therefore influence the expression of the GH1 gene in these individuals.

What is claimed:

1. A method for detecting a variation in GH1 effective to act as an indicator of GH dysfunction in an individual, wherein said method comprises the steps of:
(a) obtaining a test sample comprising a nucleotide sequence of human GH1 gene from said individual;
(b) examining the sequence obtained from the test sample to identify any test sample variants;
(c) if a test sample variant is identified, comparing the test sample variant with a mutation in Table 7B herein and where it is determined that one or more of said mutations in Table 7B is, or are, present in said test sample;
(d) concluding that said test sample variant is an effective indicator of growth hormone dysfunction wherein the test sample is obtained from an individual exhibiting the following criterion:
(i) growth failure, defined as a growth pattern which, when plotted on a standard height chart, predicts an adult height for the individual which is outside the individual's estimated target adult height range, the estimate being based upon the heights of the individual's parents.

2. The method according to claim 1, wherein the test sample is obtained from an individual exhibiting at least one of the following further criteria:
(ii) height velocity below the 25^{th} centile for age; and/or
(iii) bone age delay of at least two years when compared with chronological age; and/or
(iv) no other disorder known to cause inclusion in criteria (i) to (iii) above.

3. The method according to claim 2, wherein the bone age delay is in the range of from 2 to 4 years, when compared with chronological age.

4. The method according to claim 1, wherein the individual exhibits normal results in a standard growth hormone secretion test.

5. The method according to claim 1, wherein the method comprises any sequencing method for determining the sequence of the GH1 gene of an individual.

6. The method according to claim 1, wherein the method comprises PCR amplification of the GH1 gene of the individual using (a) a GH1 gene-specific fragment, being a fragment unique to the GH1 gene whose sequence is not found in four other paralogous genes in the GH cluster selected from the group consisting of CSH1, CSH2, CSHP1 and GH2, and (b) one or more GH1 gene-specific primers which cannot bind to homologous flanking regions in the four other paralogous non-GH1 genes in the GH cluster.

7. The method according to claim 1, wherein the method comprises PCR amplification of the entire GH1 gene of the individual and nested PCR of overlapping constituent fragments of the GH1 gene of the individual.

8. The method according to claim 1, wherein the method comprises PCR amplification of all or a fragment of genomic DNA spanning the Locus Control Region of the GH1 gene.

9. The method according to claim 1, wherein the method comprises mutational screening of all or a fragment of the individual's GH1 gene by DHPLC.

10. The detection method according to claim 1, wherein the detection method is selected from the group consisting of:
Lys 41 Arg (AAG→AGG: 731) in patient 10 (1);
Ser 108 Cys (AGC→TGC: 1023) in patient 30 (2);
Ser 108 Arg (AGC→CGC: 1023) in patient 32-2 (2);
gene conversion in patient 33 (4);
Asp Val (GAC→GTC: 477) in patient 36 (5);
IUS2 G→A in patient 37(1);
Thr 175 Ala (ACA→GCA: 1477) in patient 39 (3);
Leu −11 Pro (CTC→CCC: 369) in patient 50 (1);
Ser 71 Phe (TCC→TTC: 821) in patient 53 (1);
Gin 91 Leu (CAG→CTG: 1029) in patient 62 (2);
Glu 74 Lys (GAG→AAG: 921) in patient 63 (2); and
Val 110 Ile (GTC→ATC: 1029) in patient 66 (2).

11. A GH1 mutation comprising any one or more of said mutations in Table 7B herein.

12. A screening method for identifying an individual with GH dysfunction, wherein said screening method comprises the steps of:
(a) obtaining a test sample comprising a nucleotide sequence of the human GH1 gene from the individual; and
(b) comparing a region of the sequence obtained from the test sample with the same region of a GH1 sequence that includes at least one mutation in Table 7B; and
(c) if said test sample nucleotide sequence is shown to contain one of said mutations in Table 7B, concluding GH dysfunction is present

13. The screening method according to claim 12, wherein the test sample comprises genomic DNA.

14. A screening method for screening an individual suspected of GH dysfunction, comprising the steps of:
(a) obtaining a test sample comprising a nucleotide sequence of the human GH1 gene or an amino acid sequence encoded thereby from the individual; and
(b) analysing the test sample for the presence of a mutation of GH1 or a GH mutation or for the presence of one or more surrogate markers that are indicative of or correlated to the presence of a mutation of GH1 or a GH mutation,
wherein the mutation of GH1 or the GH mutation exhibits at least one variation when compared to the wild type hGH sequence and is obtainable from a second test sample derived from an individual exhibiting the following criterion:
(i) growth failure defined as a growth pattern which, when plotted on a standard height chart, predicts an adult height for the individual which is outside the individual's estimated target adult height range, the estimate being based upon the heights of the individual's parents.

15. The screening method according to claim 12, comprising:
(a) obtaining a transcript of said test sample nucleotide sequence; and
(b) comparing said transcript with a transcript of GH1 shown in Table 7B herein in order to determine whether any of the transcript mutations shown in Table 7B exist in said test sample transcript; and
(c) where at least one of said transcript mutations of Table 7B is found in said test sample transcript concluding GH dysfunction is present.

16. The screening method according to claim 12, wherein part (b) thereof involves comparing multiple regions of said test sample nucleotide sequence with multiple regions of said same sequences with a view to identifying at least one of the mutations in Table 7B wherein said method involves:
i) hybridisation of a labelled sample of DNA derived from the individual micro-array of variant-specific oligonucleotide probes which are immobilised on a solid support.
